SEQUENCE LISTING (4 sequences)

SEQ ID NO: 1 - DNA, 21 bp, Mus musculus
  source 1..21: mol_type = other DNA
cagatggctcggagataccac

SEQ ID NO: 2 - DNA, 22 bp, Mus musculus
  source 1..22: mol_type = other DNA
ttcccgctccaccaaggtgaag

SEQ ID NO: 3 - DNA, 25 bp, Mus musculus
  source 1..25: mol_type = other DNA
gctctgtaagggaaagagatgaccc

SEQ ID NO: 4 - DNA, 26 bp, Mus musculus
  source 1..26: mol_type = other DNA
aataggacatacccagttctgtgagg

(19) United States
(12) Patent Application Publication
(10) Pub. No.: US 2026/0325473 A1
(43) Pub. Date: Oct. 22, 2026

(54) N-Beta-Hydroxybutyryl-Amino Acids and Related Compositions and Methods

(71) Applicant: The Board of Trustees of the Leland Stanford Junior University, Stanford, CA (US)

(72) Inventors: Jonathan Z. Long, Stanford, CA (US); Veronica L. Li, Stanford, CA (US); Maria de los Dolores Moya Garzon, Stanford, CA (US); Steven M. Banik, Stanford, CA (US)

(21) Appl. No.: 19/163,706

(22) PCT Filed: Mar. 13, 2024
(86) PCT No.: PCT/US2024/019789

§ 371 Date: Sep. 9, 2025

Related U.S. Application Data
(60) Provisional application No. 63/452,060, filed on Mar. 14, 2023.
(60) Provisional application No. 63/458,684, filed on Apr. 12, 2023.

(51) Int. Cl. A61K 31/198; A61P 3/04

(52) U.S. Cl. CPC A61K 31/198; A61P 3/04

(57) ABSTRACT

N-beta-hydroxybutyryl-amino acids are provided. Also provided are methods of treating a metabolic disorder, inflammatory disorder, or muscle disorder, increasing muscle mass and/or improving muscle function, decreasing body weight and promoting health rejuvenation. Aspects of the methods include administering a therapeutically effective amount of an N-beta-hydroxybutyryl-amino acid to a subject. In some embodiments, the N-beta-hydroxybutyryl-amino acid is administered to a subject to reduce food intake, reduce body weight, reduce adipose tissue, improve glucose homeostasis, increase muscle mass, improve muscle mass, and/or promote health rejuvenation. Also provided are compositions comprising N-beta-hydroxybutyryl-amino acids.

### CROSS-REFERENCE

Pursuant to 35 U.S.C. § 119 (e), this application claims priority to the filing date of U.S. Provisional Application No. 63/452,060 filed on Mar. 14, 2023, and U.S. Provisional Application No. 63/458,684 filed on Apr. 12, 2023, the disclosures of which applications are incorporated herein by reference in their entireties.

### STATEMENT REGARDING FEDERALLY SPONSORED RESEARCH OR DEVELOPMENT

This invention was made with Government support under contract DK124265 awarded by the National Institutes of Health. The Government has certain rights in the invention.

### INCORPORATION BY REFERENCE OF SEQUENCE LISTING XML FILE

A Sequence Listing is provided herewith as a Sequence Listing XML, “STAN-2079WO_S22-312_SEQ_LIST”, created on Mar. 13, 2024 and having a size of 4,554 bytes. The contents of the Sequence Listing XML are incorporated herein by reference in their entirety.

### INTRODUCTION

Beta-hydroxybutyrate (BHB) is a fatty acid-derived ketone body produced in the liver. The classical enzymatic pathways for hepatic BHB production (ketogenesis) and extrahepatic BHB catabolismketolysis) are well-established. In the liver, fatty acid oxidation leads to the production of acetyl-CoA, which, via the sequential action of HMGCS2, HMGCL, and BDH1, ultimately results in biosynthesis of BHB. Once produced, BHB is exported via monocarboxylate transporters (MCTs) to the circulation.

Levels of circulating BHB rise during ketosis, a physiologic and metabolic state that is stimulated when carbohydrate availability is low. Once produced, BHB functions as a metabolic fuel that can be catabolized by metabolic tissues for ATP production. In recent years, systemic elevation of BHB via fasting, prolonged exercise, and/or nutritional ketosis has attracted considerable attention for potential beneficial applications in obesity, diabetes, cancer and other chronic diseases.

### SUMMARY

The present inventors reasoned that because it was known that the enzyme CNDP2 was found to produce N-lactoyl amino acids in vivo from lactate and amino acids, and beta-hydroxybutyrate (BHB) and lactate are structurally similar, that CNDP2 may also facilitate production of N-beta-hydroxybutyryl-amino acids. Using a combination of chemical syntheses and targeted metabolomics, the inventors report herein that CNDP2-dependent BHB-ylation of free amino acids is indeed a physiologic and previously unknown enzymatic reaction in vitro and in vivo. This CNDP2-dependent BHB-ylation of free amino acids represents a previously unknown extrahepatic pathway of BHB metabolism and produces a novel class of orphan metabolites, the keto amino acids (i.e., N-beta-hydroxybutyryl-amino acids). Keto amino acids are endogenously present in mouse and human plasma and exhibit physiologic and genetic regulation. Keto amino acids are a class of orphan ketone-derived metabolites linked to energy balance that exhibit various activities, including, but not limited to, hypophagic activity and anti-obesity activity.

N-beta-hydroxybutyryl-amino acids are provided. Also provided are methods of treating a metabolic disorder, inflammatory disorder, or muscle disorder, increasing muscle mass and/or improving muscle function, decreasing body weight and promoting health rejuvenation. Aspects of the methods include administering a therapeutically effective amount of an N-beta-hydroxybutyryl-amino acid to a subject. In some embodiments, the N-beta-hydroxybutyryl-amino acid is administered to a subject to reduce food intake, reduce body weight, reduce adipose tissue, improve glucose homeostasis, increase muscle mass, improve muscle function, and/or promote health rejuvenation. Also provided are compositions comprising N-beta-hydroxybutyryl-amino acids.

### BRIEF DESCRIPTION OF THE FIGURES

FIGS. 1A-1G. CNDP2 catalyzes amino acid BHB-ylation in vitro. (FIG. 1A) Chemical structure of lactate (C3 hydroxy acid, top) and beta-hydroxybutyrate (C4 hydroxy acid, bottom). (FIG. 1B) Schematic of the CNDP2-catalyzed condensation of BHB and amino acids to form keto amino acids. (FIG. 1C) Keto-Phe synthase activity of cell lysates of HEK293T cells transfected with GFP, CNDP2-WT or catalytically dead mutant CNDP2-E166A and treated with BHB (20 mM) and phenylalanine (20 mM). (FIG. 1D) Background-subtracted CNDP2 synthase activity of cell lysates of CNDP2-transfected HEK293T cells towards acetate, lactate, BHB and octanoate. All organic acid substrates were tested at 1 mM concentration with 10 mM phenylalanine. (FIG. 1E) Background-subtracted Keto amino acid synthase activity of cell lysates of HEK293T cells transfected with CNDP2 and treated with BHB (20 mM) and amino acids (20 mM). (FIG. 1F) Keto amino acids quantitation in conditioned media from HEK293T cells transfected with GFP (white) or CNDP2 (black) and supplemented with 25 mM BHB overnight. (FIG. 1G) Percentage Keto-Phe, Keto-Val and Keto-Leu in conditioned media (extracellular) or cell lysates (intracellular) from HEK293T cells transfected with CNDP2 and incubated with 25 mM BHB overnight. (FIGS. 1H-11) Rate of BHB-Phe (FIG. 1H) or Lac-Phe (FIG. 11) production following incubation of purified recombinant mouse CNDP2 with the indicated concentration of BHB or lactate and 20 mM phenylalanine. For FIGS. 1C-1D, N=3 per group; for FIGS. 1E-1F, N=4 per group; for FIGS. 1H-11, N=3 per group. P values were calculated by Student's two-sided t-test.

FIGS. 2A-2D. Additional in vitro characterization of CNDP2 activity. (FIGS. 2A-2B) Molecular docking simulations of human CNDP2 with BHB (FIG. 2A) or lactate (FIG. 2B) in the active site. Side chains of the active site binding pocket are shown along with two Mn2+ ions in purple. (FIG. 2C) Western blotting using the indicated antibodies for HEK293T cells transfected with either CNDP2-flag or mutant CNDP2 E166A-flag. (FIG. 2D) BHB-Phe synthase activity of cell lysates of HEK293T cells mock-transfected or transfected with CNDP2 and treated with BHB and phenylalanine, 2-HB and phenylalanine or 3-HIB and phenylalanine. Organic acids and phenylalanine were incubated at the indicated concentrations. For FIG. 2D, N=4 per group. Data are shown as means±SEM. P values were calculated by Student's two-sided t-test.

FIGS. 3A-3G. CNDP2 catalyzes amino acid BHB-ylation in mouse tissues. (FIG. 3A) Western blot across mouse tissues using an anti-CNDP2 (top) or anti-tubulin (bottom) antibody. (FIGS. 3B-3G) Enzyme activities of tissues from WT or CNDP2-KO mice when provided with 20 mM BHB and 20 mM Phe (FIG. 3B), 20 mM BHB and 20 mM Leu (FIG. 3C), 20 mM BHB and 20 mM Val (FIG. 3D), 20 mM BHB and 20 mM Met (FIG. 3E), 20 mM lactate and Phe (FIG. 3F), or 10 mM carnosine (FIG. 3G) as substrates. For FIGS. 3B-3G, N=3-4 per group. Data are shown as means±SEM. P values were calculated by Student's two-sided t-test.

FIGS. 4A-4C. Additional characterization of CNDP2 BHB-ylation activity. (FIG. 4A) Western blot across WT and CNDP2-KO mouse tissues using an anti-CNDP2 (top) or anti-tubulin (bottom) antibody. (FIGS. 4B-4C) Temperature dependance of BHB-Phe synthesis activity of kidney lysate (FIG. 4B) or recombinant CNDP2 protein (FIG. 4C). For FIGS. 4B-4C, N=3 per group. Data are shown as means±SEM.

FIGS. 5A-5F. Detection and dynamic regulation of keto amino acids in mouse plasma. (FIGS. 5A-5D) Tandem mass spectrometry fragmentation of an authentic standard (left) and co-elution of the standard with the endogenous peak from mouse plasma using a multiple reaction monitoring method with the indicated transition (right) for Keto-Phe (FIG. 5A), Keto-Leu (FIG. 5B), Keto-Val (FIG. 5C), and Keto-Met (FIG. 5D). (FIGS. 5E-5F) BHB (FIG. 5E) and keto amino acid (FIG. 5F) quantitation in 8-9 week male C57BL/6J mouse plasma at baseline (dark blue), after 1 week on ketogenic diet (89% of calories from fat, 10% of calories from protein, 1% of calories from carbohydrates, red), after a 24 h fast (grey) or 30 min post ketone monoester drink administration by oral gavage (KetoneAid KE4 Ketone Ester Performance Drink, 3 mg KE/g of body weight, light blue). For FIGS. 5E-5F, N=4-5 per group. Data are shown as the mean±s.e.m. P values were calculated by Student's two-sided t-test.

FIGS. 6A-6C. Comparison of fragmentation patterns for BHB-Phe, 2-HB-Phe, and 3-HIB-Phe._(FIGS. 6A-6B) Structure (left) and tandem mass spectrometry fragmentation (right) and (FIG. 6C) relative abundances for the indicated MRM transitions for the indicated BHB-Phe, 2-HB-Phe, and 3-HIB-Phe standards and for mouse plasma.

FIGS. 7A-7F. Biochemical and cellular organization of amino acid BHB-ylation, lactate, ketolysis pathways in vivo. (FIG. 7A) Schematic showing the interplay of CNDP2-dependent keto or lactoyl amino acid biosynthesis. (FIG. 7B) Background-subtracted Keto-Phe (black) and Lac-Phe (white) synthase activity of cell lysates of HEK293T cells transfected with CNDP2 and incubated with BHB and/or lactate at the indicated concentrations and with phenylalanine (20 mM). (FIG. 7C) Relative fold change of Keto-Phe and Lac-Phe in lean mice after 15 min of running (“acute running”), 30 min after consumption of a ketone drink (3 mg ketone ester/g of body weight, “ketone drink”), or after 30 min of strenuous running (“prolonged running”). (FIG. 7D) Clustered heat map of Z-scores for the gene expression of Oxct1, Cndp2, and Hmgcl across 81 cell types in Tabula muris. (FIG. 7E) Keto amino acid levels in media from human induced pluripotent stem cell-derived cardiomyocytes or RAW264 macrophages after treatment with BHB (25 mM, overnight). (FIG. 7F) Schematic of classical ketogenesis and ketolysis and non-classical keto amino acid production by BHB-ylation of amino acids. For FIG. 7B, N=4 per group, for FIG. 7C, N=5 per group, for FIG. 7E, N=4/group. Data are shown as the mean±s.e.m. P values were calculated by Student's two-sided t-test.

FIGS. 8A-8E. Genetic and enzymatic regulation of keto amino acids. (FIG. 8A) Schematic of genetic mouse models used in this study that disrupt either ketogenesis or amino acid BHB-ylation. (FIGS. 8B-8C) Keto amino acid quantitation in plasma from male and female Hmgc/(fl/fl) (white) vs Alb-Hmgcl (−/−) mice (black) after a 24 h fast (FIG. 8B) or two days on ketogenic diet (89.5% of calories from fat, 10.4% of calories from protein, 0.1% of calories from carbohydrates) (FIG. 8C). (FIGS. 8D-8E) Keto amino acid quantitation in plasma from 7-30 week male/female WT (white) and CNDP2-KO mice (black) at 60 min post ketone monoester drink administration by oral gavage (3 mg KE/g of body weight) (FIG. 8D) or after a 24 h fast (FIG. 8E). For FIG. 8B, N=5 per group; for FIG. 8C, N=3-4 per group; for FIG. 8D, N=3-6 per group; for FIG. 8E, N=5 per group. Data are shown as the mean±s.e.m. P values were calculated by Student's two-sided t-test.

FIG. 9. BHB quantitation in plasma from 7-16-week-old female WT and CNDP2-KO mice after 1 week on ketogenic diet (Research Diets D06040601).

FIGS. 10A-10D. Identification of direct protein targets for keto amino acids. (FIG. 10A) Chemical structure of the X-Phe photoprobe (top) and Keto-Phe (bottom). (FIG. 10B) Schematic of the chemoproteomics approach with X-Phe photoprobe in mouse whole tissue homogenized lysates. (FIG. 10C) TAMRA in-gel fluorescence of whole tissue homogenized lysates (brain, heart, quadriceps, and liver) treated with X-Phe photoprobe (100 UM) and conjugated with rhodamine azide fluorophore via click chemistry for in-gel visualization. (FIG. 10D) Quantitative shotgun proteomics of brain lysate proteins crosslinked by X-Phe photoprobe (100 μM) and competed with either Keto-Phe or Lac-Phe (50-fold excess competitor).

FIGS. 11A-11B. Proteins crosslinked by X-Phe photoprobe and competed >80% by both BFB-Phe and Lac-Phe. FIG. 11A shows Proteinld, gene symbol, and description. FIG. 11B shows Proteinld, Probe 127C, Probe_50xLac-Phe 128C, Probe_50xBHB-Phe 129N, functional evidence in obesity, reference (PMID), genetic evidence in obesity, and T2D AMP link.

FIGS. 12A-12K. BHB-amino acids suppress food intake and body weight. (FIGS. 12A-12E) Food intake (FIG. 12A), ambulatory movement (FIG. 12B), oxygen consumption (VO2) (FIG. 12C), carbon dioxide production (VCO₂) (FIG. 12D), and respiratory exchange ratio (RER) (FIG. 12E) of 29-week-old male DIO mice following a single injection of vehicle or BHB-Phe (50 mg/kg, IP) over a 10 h period in metabolic chambers. (FIGS. 12F-12G) Change in body weight (FIG. 12F) and cumulative food intake (FIG. 12G) of 28-week-old male DIO mice treated with vehicle or BHB-Phe (50 mg/kg/day, IP). (FIG. 12H) Change in body weight (left) and daily food intake (right) of 15-week-old male DIO mice after 6 days of treatment with vehicle, BHB-Phe (50 mg/kg/day, IP) or vehicle-treated pair-fed mice. (FIG. 121) Daily food intake (left) and change in body weight (right) of 13-week-old male DIO mice after 9 days of treatment with vehicle, BHB-Phe (50 mg/kg/day, IP) or equivalent doses of BHB or phenylalanine alone. (FIGS. 12J-12K) Change in body weight (FIG. 12J) and food intake (FIG. 12K) of 14-16-week-old male DIO mice after 9 days of treatment with BHB-Phe, BHB-Lys, BHB-His, Phe-Phe, Leu-Leu or vehicle (50 mg/kg/day, IP). For FIGS. 12A-12E, N=7 for vehicle, N=8 for BHB-Phe. For FIGS. 12F-12G, N=10 per group. For FIGS. 12H-12K, N=5 per group. Data are shown as the mean±SEM. P values were calculated by Student's two-sided t-test or by two-way ANOVA.

FIG. 13A-13E. Additional characterization of mice treated with BHB-Phe. (FIG. 13A) Food intake (left) and plasma BHB-Phe levels (right) of 14-week-old male DIO mice following a single injection of BHB-Phe (50 mg/kg, IP) over a 3 h period. (FIG. 13B) Food intake (left), kaolin intake (middle) and water intake (right) of 16-week-old male DIO mice following a single injection of vehicle or BHB-Phe (50 mg/kg, IP). (FIG. 13C) Plasma acyl-ghrelin (left), leptin (middle), and GDF-15 (right) of 18-week-old male DIO mice 1 hour after a single injection of vehicle or BHB-Phe (50 mg/kg, IP). (FIG. 13D) Plasma AST, ALT, TG, HDL, LDL after 14 days of treatment with BHB-Phe (50 mg/kg/day, IP) or vehicle. (FIG. 13E) Change in body weight (left) and daily food intake (right) of 15-week-old male DIO mice after 5 days of treatment with vehicle, BHB-Phe, BHB-Leu, BHB-Val or BHB-Met (50 mg/kg/day, IP). For FIG. 13A, N=5 per group. For FIG. 13B, N=9 per group. For FIG. 13C, N=5 for vehicle and N=4-5 for BHB-Phe. For FIG. 13D, N=8 per group. For FIG. 13E, N=5 per group. Data are shown as the mean±SEM. P values were calculated by Student's two-sided t-test.

FIGS. 14A-14D. Conservation and ketosis-inducible keto amino acids in human plasma. (FIGS. 14A-14B) Quantification of BHB (FIG. 14A) and keto amino acids (FIG. 14B) in human plasma at the indicated time point with a ketone drink (3 mg KE/g of body weight). (FIGS. 14C-14D) Quantification of BHB (FIG. 14C) and BHB-amino acids (FIG. 14D) at baseline or 4 weeks after WFKD intervention. For FIGS. 14A-14B, N=7 per group, for FIGS. 14C-14D, N=12 per group. Data are shown as the mean±SEM. P values were calculated by Student's two-sided t-test. FIG. 15. Multiple reaction monitoring (MRM) parameters for metabolites measured.

### DETAILED DESCRIPTION

N-beta-hydroxybutyryl-amino acids are provided. Also provided are methods of treating a metabolic disorder, inflammatory disorder, or muscle disorder, increasing muscle mass and/or improving muscle function, decreasing body weight and promoting health rejuvenation. Aspects of the methods include administering a therapeutically effective amount of an N-beta-hydroxybutyryl-amino acid to a subject. In some embodiments, the N-beta-hydroxybutyryl-amino acid is administered to a subject to reduce food intake, reduce body weight, reduce adipose tissue, improve glucose homeostasis, increase muscle mass, improve muscle mass, and/or promote health rejuvenation. Also provided are compositions comprising N-beta-hydroxybutyryl-amino acids.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. It is understood that the present disclosure supersedes any disclosure of an incorporated publication to the extent there is a contradiction. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. It must be noted that as used herein and in the appended claims, the singular forms “a”, “an”, and “the” include plural referents unless the context clearly dictates otherwise. For example, the term “an agent” refers to one or more agents, i.e., a single agent and multiple agents. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### N-Beta-Hydroxybutyryl-Amino Acids

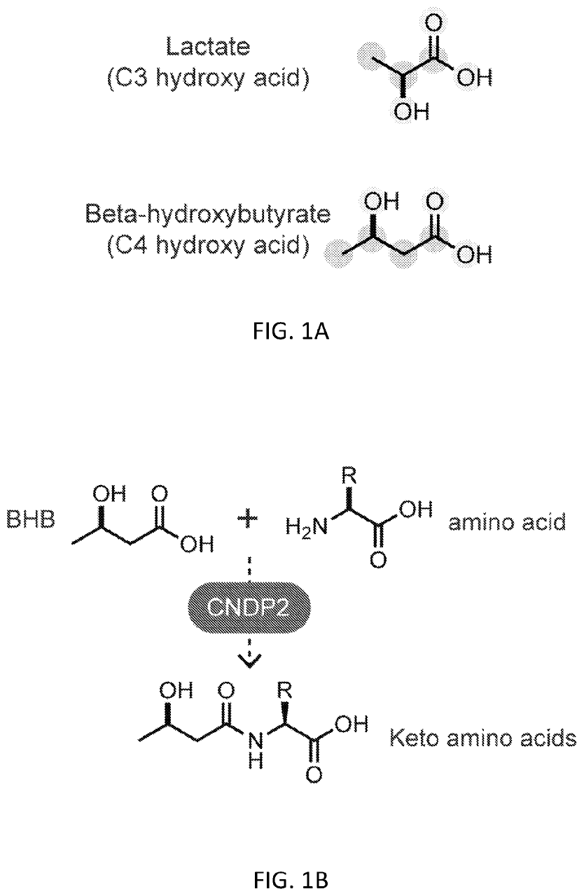

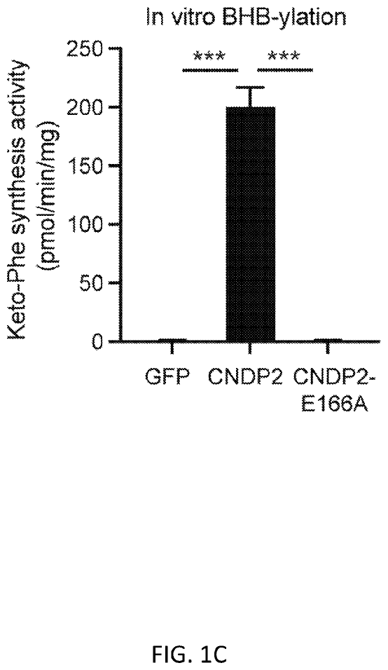

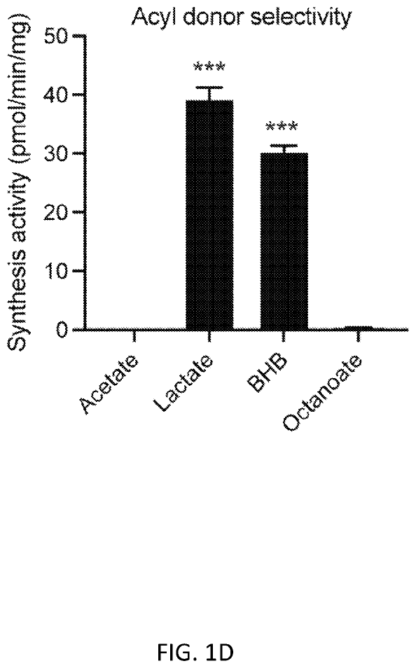

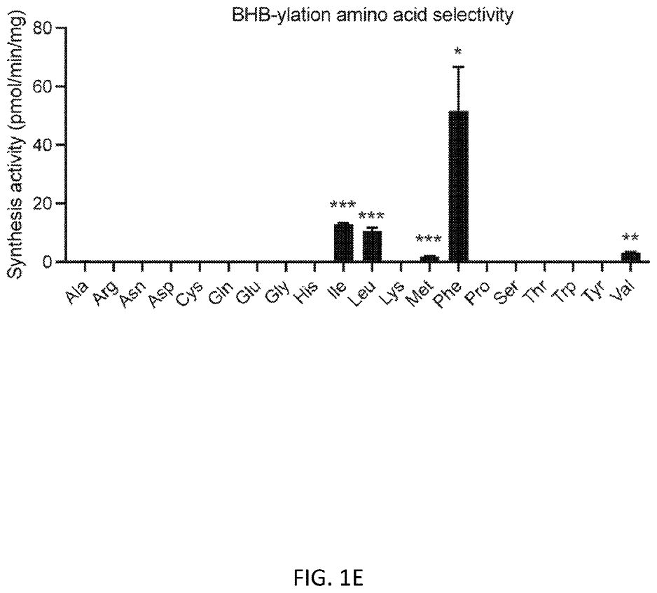

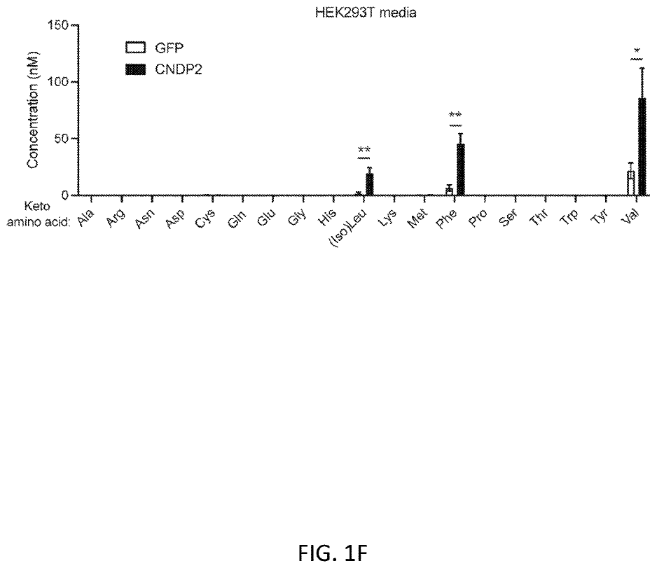

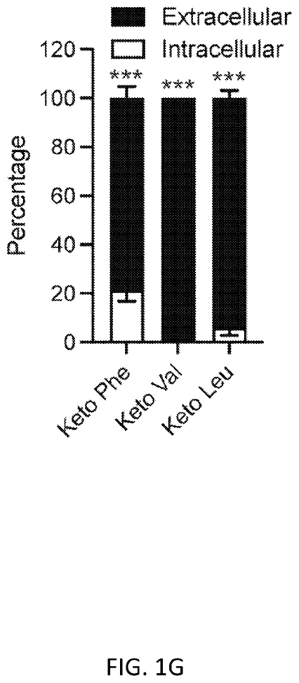

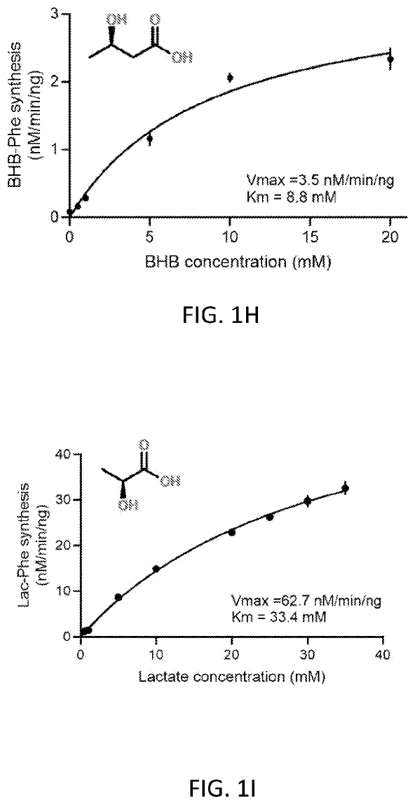

As reviewed above, N-beta-hydroxybutyryl-amino acids are provided. N-beta-hydroxybutyryl-amino acids are amino acids modified with a beta-hydroxybutyryl group, wherein the amino group of the amino acid is conjugated to the carboxyl group of beta-hydroxybutyrate (BHB) (See, e.g., FIG. 1B). The terms “N-beta-hydroxybutyryl-amino acid”, “BHB-amino acid” and “keto amino acid” are used interchangeably herein.

Beta-hydroxybutyrate (BHB) is a chiral compound and thus has two enantiomers: D-BHB and L-BHB. In certain embodiments, the N-beta-hydroxybutyryl-amino acid comprises a D-beta-hydroxybutyryl group. In certain embodiments, the N-beta-hydroxybutyryl-amino acid comprises an L-beta-hydroxybutyryl group. Amino acids exist in both L enantiomers and D enantiomers. In certain embodiments, the N-beta-hydroxybutyryl-amino acid comprises a D-amino acid. In certain embodiments, the N-beta-hydroxybutyryl-amino acid comprises an L-amino acid. In certain embodiments, the N-beta-hydroxybutyryl-amino acids comprise a mixture of enantiomers and/or stereoisomers. In certain embodiments, the N-beta-hydroxybutyryl-amino acids comprise a single enantiomer or stereoisomer. In certain embodiments, the N-beta-hydroxybutyryl-amino acid comprises a D-beta-hydroxybutyryl group and an L-amino acid.

N-beta-hydroxybutyryl-amino acids may comprise coded and non-coded amino acids. N-beta-hydroxybutyryl-amino acids may comprise chemically or biochemically modified amino acids and/or derivatized amino acids. Amino acids include, but are not limited to, glycine, alanine, serine, threonine, cysteine, valine, leucine, isoleucine, methionine, proline, phenylalanine, tyrosine, tryptophan, aspartic acid, glutamic acid, asparagine, glutamine, histidine, lysine, and arginine.

In some embodiments, N-beta-hydroxybutyryl-amino acids comprise an amino acid with a hydrophobic side chain. Amino acids with hydrophobic side chains have little or no polarity in their side chains. Amino acids with hydrophobic side chains include, but are not limited to, glycine, alanine, valine, leucine, isoleucine, proline, phenylalanine, methionine, and tryptophan. In some embodiments, the N-beta-hydroxybutyryl-amino acid is N-beta-hydroxybutyryl-phenylalanine (BHB-Phe), N-beta-hydroxybutyryl-leucine (BHB-Leu), N-beta-hydroxybutyryl-isoleucine (BHB-Ile), N-beta-hydroxybutyryl-valine (BHB-Val), or N-beta-hydroxybutyryl-methionine (BHB-Met). In some embodiments, the N-beta-hydroxybutyryl-amino acid is N-beta-hydroxybutyryl-phenylalanine (BHB-Phe). In some embodiments, the N-beta-hydroxybutyryl-amino acid is N-beta-hydroxybutyryl-leucine (BHB-Leu). In some embodiments, the N-beta-hydroxybutyryl-amino acid is N-beta-hydroxybutyryl-isoleucine (BHB-Ile). In some embodiments, the N-beta-hydroxybutyryl-amino acid is N-beta-hydroxybutyryl-valine (BHB-Val). In some embodiments, the N-beta-hydroxybutyryl-amino acid is N-beta-hydroxybutyryl-methionine (BHB-Met). In some embodiments, N-beta-hydroxybutyryl-amino acids comprise a single type of N-beta-hydroxybutyryl-amino acids (e.g., BHB-Phe). In some embodiments, N-beta-hydroxybutyryl-amino acids comprise a mixture of N-beta-hydroxybutyryl-amino acids (e.g., a mixture of BHB-Phe and BHB-Leu). The mixture of N-beta-hydroxybutyryl-amino acids may comprise a mixture of any of the N-beta-hydroxybutyryl-amino acids listed above in any ratio.

N-hydroxybutyryl-amino acids may be naturally occurring (e.g., endogenous) or synthetic. N-hydroxybutyryl-amino acids may be endogenously present in mammals, including, but not limited to humans, mice and rats. N-hydroxybutyryl-amino acids may be produced from BHB and an amino acid via a BHB-ylation reaction. The BHB-ylation reaction may be carried out enzymatically or chemically. In certain embodiments, the BHB-ylation is carried out by the enzyme CNDP2. N-hydroxybutyryl-amino acids may be synthesized chemically. For example, methods of chemically synthesizing N-beta-hydroxybutyryl-amino acids may include well known chemistries and amino acid conjugation chemistries known in the art.

In some embodiments, the N-beta-hydroxybutyryl-amino acid of the present invention exhibits physiological activity. By physiological activity it is meant that the N-beta-hydroxybutyryl-amino acid exhibits a measurable biological response and/or change. Biological responses include, but are not limited to, molecular responses, cellular responses, tissue-specific responses, organ-specific responses, organism-specific responses and combinations thereof. Measurable biological responses are able to be detected and/or quantified as a change from a baseline amount and/or baseline activity.

Physiological activity may include hypophagic activity. Hypophagia is defined as the reduced ingestion of food. Hypophagic activity is characterized by decreased ingestion of food, decreased consumption of food, loss of appetite, not feeling hungry, and combinations thereof. In some embodiments, the N-beta-hydroxybutyryl-amino acid exhibits hypophagic activity. In some embodiments, the N-beta-hydroxybutyryl-amino acid exhibits human hypophagic activity. In some embodiments, the N-beta-hydroxybutyryl-amino acid exhibits murine hypophagic activity.

### METHODS OF USE

As reviewed above, the present disclosure includes methods of treating a metabolic disorder, inflammatory disorder, or muscle disorder, increasing muscle mass and/or improving muscle function, decreasing body weight and promoting health rejuvenation. Aspects of such methods include administering an N-hydroxybutyryl-amino acid to a subject.

The terms “individual”, “subject”, and “recipient”, are used interchangeably herein and refer to any mammalian subject for whom diagnosis, treatment, therapy, or effective change or maintenance (e.g., a decrease in or maintenance of food intake) is desired, particularly humans. In some embodiments, the mammal is a human. Subjects may be human, but also include other mammals, particularly those mammals useful as laboratory models for human disease, e.g., mice, rats, etc.

In some embodiments, the method is a method of treating or administering an effective amount of an agent (e.g., an N-beta-hydroxybutyryl-amino acid) to a subject, wherein the subject is a mammal. In some embodiments, the method is a method of treating or administering an effective amount of an agent (e.g., an N-beta-hydroxybutyryl-amino acid) to a subject, wherein the subject is a human. In some embodiments, the method is a method of treating or administering an effective amount of an agent (e.g., an N-beta-hydroxybutyryl-amino acid) to a subject, wherein the subject is an adult. The term “adult” is used to describe a subject (e.g., a human) that is fully grown and/or developed. In general, humans that are adults are age eighteen or older.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with a metabolic disorder) as well as those in which prevention is desired (e.g., those with a genetic predisposition for developing a metabolic disorder, those with increased susceptibility to a metabolic disorder, those with an increased likelihood of a metabolic disorder, those suspected of having a metabolic disorder, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, an “effective amount” refers to that amount of the agent sufficient to induce the claimed effect (e.g., the amount of the agent sufficient to reduce food intake, reduce body weight, increase glucose clearance, increase muscle mass, improve muscle function, reduce inflammation and rejuvenate health). Further, an effective amount with respect to an agent of the invention means the amount of agent alone, or in combination with other agents, that provide the claimed effect.

### Methods of Treating Metabolic Disorders

In some embodiments, the method is a method of treating a subject for a metabolic disorder. Metabolism refers to the whole sum of reactions that are responsible for providing the body with energy. Metabolic disorders generally refer to a broad array of disorders characterized by defects that interfere with the body's metabolism (i.e., the chemical processes by which a body transforms proteins, carbohydrates and fats into energy). For example, metabolic disorders can affect the body's ability to synthesize, process, distribute, and/or breakdown macronutrients, such carbohydrates, proteins, nucleic acids, and lipids and other essential nutrients including, but not limited to, iron and calcium.

Types of metabolic disorders include, but are not limited to, glucose metabolism disorders, iron metabolism disorders, lipid metabolism disorders, malabsorption syndromes, mitochondrial disorders and proteostasis disorders. Metabolic disorders include obesity, obesity related disorders, diabetes, Gaucher disease, Krabbe disease, maple syrup urine disease, hemochromatosis, phenylketonuria, mitochondrial disease, porphyria, Fabry disease, dyslipidemia, hypertension, malabsorption syndrome, organic acidemias, metachromatic leukodystrophy, Hunter syndrome, Niemann-Pick disease, Tay-Sachs disease, Wilson's disease and urea cycle disorders.

In some embodiments, the method is a method of treating a subject for obesity or an obesity-related disorder. Obesity is defined as an excess of body fat relative to lean body mass, and is a serious contributor to increased morbidity and mortality. Obesity, which is most commonly caused by excessive food intake coupled with limited energy expenditure and/or lack of physical exercise, often accompanies various glucose metabolism disorders. A subject is generally defined as obese if the subject has a body mass index of 30 kg/m² or greater. Obesity increases the likelihood of various disorders. Obesity-related disorders may include, but are not limited to, hypertension, dyslipidemia, mellitus, atherosclerosis, gout, rheumatism, arthritis, type 2 diabetes, coronary heart disease, stroke, gallbladder disease, liver disease, sleep apnea and pain. Obesity is often associated with psychological and medical morbidities, the latter of which includes increased joint problems, vascular diseases such as coronary artery disease, hypertension, stroke, and peripheral vascular disease. Obesity also causes metabolic abnormalities such as insulin resistance and Type II diabetes (non-insulin-dependent diabetes mellitus (NIDDM)), hyperlipidemia, and endothelial dysfunction.

In some embodiments, the method is a method of treating a subject for diabetes. Diabetes is a disorder wherein the body does not make enough insulin or cannot effectively use insulin. Insulin allows sugar (i.e., glucose) to be released from the bloodstream and into cells for use as energy. As a result, diabetes causes too high levels blood sugar and poor glucose clearance from the blood. Diabetes includes type 1 diabetes and type 2 diabetes.

### Methods of Treating Inflammatory Disorders

In some embodiments, the method is a method of treating a subject for an inflammatory disorder. Inflammation is a biological response to harmful stimuli such as pathogens, damaged cells, or irritants. As such, a disorder of inflammation (i.e. an inflammatory disorder) is one wherein inflammation is dysregulated within the organism. Inflammation can present as heat, pain, redness, swelling and/or loss of function. Inflammation leads to a shift in the type of cells and/or signaling molecules present at the inflammation site. For example, inflammation may result in an increase of leukocytes and cytokines at the inflammation site. Inflammatory disorders include disorders wherein too little inflammation occurs and disorders wherein too much inflammation occurs. Inflammatory disorders include acute inflammatory disorders (i.e., the initial response to harmful stimuli) and chronic inflammatory disorders (i.e. prolonged inflammation). Acute inflammation may last for hours to days. Acute inflammatory diseases include, but are not limited to, ileus and appendicitis. Chronic inflammation may last for one month to one year, or even longer than a year. Chronic inflammatory disorders include, but are not limited to, irritable bowel syndrome, inflammatory bowel disease, Crohn's disease, asthma, colitis and fibrosis.

### Methods of Treating Muscle Disorders

In some embodiments, the method is a method of treating a muscle disorder. Muscle disorders are disorders that affect the muscles in the body, including, but not limited to, skeletal muscles. Skeletal muscles are muscles that are attached to bones and responsible for skeletal movement in vertebrates. Muscle disorders may result in weakness, fatigue, pain, numbness, and/or paralysis of muscles. Muscle disorders may result in muscle loss, movement issues, and/or balance problems. Muscle disorders include fibromyalgia, movement disorders, multiple sclerosis, muscle cramps, muscular dystrophy, myasthenia gravis, myositis, and neuromuscular disorders.

### Methods of Reducing Food Intake, Reducing Body Weight, Increasing Glucose Clearance, Increasing Muscle Mass, Improving Muscle Function, Reducing Inflammation and Rejuvenating Health

As reviewed above, in some embodiments, the N-beta-hydroxybutyryl-amino acid of the present invention exhibits physiological activity (i.e., a measurable physiological change) when administered to a subject. In some embodiments, administration of the N-hydroxybutyryl-amino acids of the present invention result in physiological activity in the subject. In some embodiments, the method results in reduced food intake, reduced body weight, increased glucose clearance, reduced adipose tissue mass, increased muscle mass, reduced inflammation, rejuvenated health and/or combinations thereof.

In some embodiments, the method reduces food intake by the subject. In some embodiments, the method reduces food intake by the subject compared to food intake by the subject before treatment and/or administration of the N-beta-hydroxybutyryl-amino acid. In some embodiments, the method reduces food intake by the subject compared to food intake by a control subject to which treatment and/or an effective amount of N-beta-hydroxybutyryl-amino acid has not been administered to. By reduced food intake, it is meant that the subject exhibits a decreased ingestion and/or consumption of food. The reduced food intake may be referred to as “hypophagia.” The reduced food intake may be a reduced cumulative intake of food and/or a reduced average daily intake of food.

By cumulative food intake, it is meant that the food intake over a period of time (e.g., the duration of the treatment or the duration of N-beta-hydroxybutyryl-amino acid administration) is measured. Cumulative food intake may be measured over a period of one hour or more, e.g., one hour, two hours, three hours, four hours, five hours, six hours, seven hours, eight hours, nine hours, ten hours, twelve hours, fourteen hours, sixteen hours, eighteen hours, twenty hours, twenty two hours, twenty four hours, etc., or two days of more, e.g., two days, three days, four days, five days, six days, seven days, eight days, nine days, ten days, etc., or one week or more, e.g., one week, two weeks, three weeks, four weeks, five weeks, six weeks, etc., or one month or more, e.g., one month, two months, three months, four months, six months, eight months, ten months, twelve months, etc., or one year or more, e.g., one year, two years, three years, four years, five years, six years, seven years, eight years, nine years, ten years, etc. In some embodiments, cumulative food intake is reduced by 1% to 99%, e.g., 5% to 95%, 10% to 90%, 10% to 70%, 10% to 50%, 15% to 50%, 15% to 40%, etc. In some embodiments, cumulative food intake is reduced by at least 1%, e.g., at least 2%, at least 5%, at least 10%, at least 15%, at least 20%, at least 25%, at least 30%, at least 35%, at least 40%, at least 45%, at least 50%, etc.

Cumulative food intake can be measured by counting the caloric intake of the subject over a period of time (e.g., the duration of the treatment or the duration of N-beta-hydroxybutyryl-amino acid administration). The cumulative food intake over a period of time of a subject to which an effective amount of an N-beta-hydroxybutyryl-amino acid has been administered may be compared to the cumulative food intake over the same period of time of the same subject prior to N-hydroxybutyryl-amino acid administration to determine if cumulative food intake has been reduced. The cumulative food intake over a period of time of a subject to which an effective amount of an N-beta-hydroxybutyryl-amino acid has been administered may be compared to the cumulative food intake over the same period of time of a control subject to which an effective amount of an N-beta-hydroxybutyryl-amino acid has not been administered to determine if cumulative food intake has been reduced.

By average daily food intake, it is meant that the food intake is measured per day and then averaged over a time period (e.g., the duration of the treatment or the duration of N-beta-hydroxybutyryl-amino acid administration). Average daily food intake may be measured over a period of two days or more, e.g., two days, three days, four days, five days, six days, seven days, eight days, nine days, ten days, etc., or one week or more, e.g., one week, two weeks, three weeks, four weeks, five weeks, six weeks, etc., or one month or more, e.g., one month, two months, three months, four months, six months, eight months, ten months, twelve months, etc., or one year or more, e.g., one year, two years, three years, four years, five years, six years, seven years, eight years, nine years, ten years, etc. In some embodiments, average daily food intake is reduced by 1% to 99%, e.g., 5% to 95%, 10% to 90%, 10% to 70%, 10% to 50%, 15% to 50%, 15% to 40%, etc. In some embodiments, average daily food intake is reduced by at least 1%, e.g., at least 2%, at least 5%, at least 10%, at least 15%, at least 20%, at least 25%, at least 30%, at least 35%, at least 40%, at least 45%, at least 50%, etc.

Daily food intake can be measured by counting the caloric intake of the subject over the course of one day (e.g., 24 hours). Average daily food intake is calculated by averaging the daily food intake of a subject over a period of time (i.e., a certain number of days). The average daily food intake of a subject to which an effective amount of an N-beta-hydroxybutyryl-amino acid has been administered may be compared to the average daily food intake of the same subject prior to N-hydroxybutyryl-amino acid administration to determine if cumulative food intake has been reduced. The average daily food intake of a subject to which an effective amount of an N-beta-hydroxybutyryl-amino acid has been administered may be compared to the average daily food intake of a control subject to which an effective amount of an N-beta-hydroxybutyryl-amino acid has not been administered to determine if cumulative food intake has been reduced. In some embodiments, the method reduces body weight of the subject compared to body weight of the subject before treatment. In some embodiments, body weight is reduced by 1% to 90%, e.g., 1% to 70%, 1% to 50%, 1% to 30%, 5% to 50%, 5% to 30%, 10% to 30%, etc. In some embodiments, body weight of the subject is reduced by at least 1%, e.g., at least 2%, at least 5%, at least 10%, at least 15%, at least 20%, at least 25%, at least 30%, at least 35%, at least 40%, at least 45%, at least 50%, etc.

In some embodiments, the method increases glucose clearance compared to glucose clearance of the subject before treatment. Glucose clearance refers to the body's ability to remove glucose from the blood. An increase in glucose clearance means the rate that the body removes glucose from the blood is increased. In some embodiments, glucose clearance in the subject is increased by at least 1%, e.g., at least 2%, at least 3%, at least 4%, at least 5%, at least 10%, at least 15%, at least 20%, at least 25%, at least 30%, at least 35%, at least 40%, at least 45%, at least 50%, at least 70%, at least 90% etc.

In some embodiments, the method reduces adipose tissue mass compared to adipose tissue mass in the subject before treatment. Adipose tissue (i.e., body fat or fat) is a tissue composed mostly of adipocytes. Adipose tissue serves as a reserve of lipids in the body. In some embodiments, adipose tissue mass is reduced by 1% to 99%, e.g., 5% to 95%, 10% to 90%, 10% to 70%, 10% to 50%, 20% to 50%, 30% to 50%, 15% to 40%, etc. In some embodiments, adipose tissue mass is reduced by at least 1%, e.g., at least 2%, at least 5%, at least 10%, at least 15%, at least 20%, at least 25%, at least 30%, at least 35%, at least 40%, at least 45%, at least 50%, etc.

The adipose tissue may be white adipose tissue and brown adipose tissue. White adipose tissue stores energy and each white adipocyte contains a single lipid droplet, while brown adipose tissue generates body heat and each brown adipocyte contains numerous lipid droplets. In some embodiments, the method reduces white adipose tissue (i.e., white fat) in the subject. In some embodiments, white fat is reduced by 1% to 99%, e.g., 5% to 95%, 10% to 90%, 10% to 70%, 10% to 50%, 20% to 50%, 30% to 50%, 15% to 40%, etc. In some embodiments, white fat is reduced by at least 1%, e.g., at least 2%, at least 5%, at least 10%, at least 15%, at least 20%, at least 25%, at least 30%, at least 35%, at least 40%, at least 45%, at least 50%, etc. In some embodiments, the method reduces brown adipose tissue (i.e., brown fat) in the subject. In some embodiments, brown fat is reduced by 1% to 99%, e.g., 5% to 95%, 10% to 90%, 10% to 70%, 10% to 50%, 20% to 50%, 30% to 50%, 15% to 40%, etc. In some embodiments, brown fat is reduced by at least 1%, e.g., at least 2%, at least 5%, at least 10%, at least 15%, at least 20%, at least 25%, at least 30%, at least 35%, at least 40%, at least 45%, at least 50%, etc.

The adipose tissue may be inguinal adipose tissue or epididymal adipose tissue. In some embodiments, the method reduces inguinal adipose tissue (i.e., inguinal fat) in the subject. In some embodiments, inguinal fat is reduced by 1% to 99%, e.g., 5% to 95%, 10% to 90%, 10% to 70%, 10% to 50%, 20% to 50%, 30% to 50%, 15% to 40%, etc. In some embodiments, inguinal fat is reduced by at least 1%, e.g., at least 2%, at least 5%, at least 10%, at least 15%, at least 20%, at least 25%, at least 30%, at least 35%, at least 40%, at least 45%, at least 50%, etc. In some embodiments, the method reduces epididymal adipose tissue (i.e., epididymal fat) in the subject. In some embodiments, epididymal fat is reduced by 1% to 99%, e.g., 5% to 95%, 10% to 90%, 10% to 70%, 10% to 50%, 20% to 50%, 30% to 50%, 15% to 40%, etc. In some embodiments, epididymal fat is reduced by at least 1%, e.g., at least 2%, at least 5%, at least 10%, at least 15%, at least 20%, at least 25%, at least 30%, at least 35%, at least 40%, at least 45%, at least 50%, etc.

In some embodiments, the method increases muscle mass compared to muscle mass in the subject before treatment. In some embodiments, muscle mass in the subject is increased by 1% to 90%, e.g., 1% to 70%, 1% to 50%, 1% to 30%, 5% to 50%, 5% to 30%, 10% to 30%, etc. In some embodiments, muscle mass in the subject is increased by at least 1%, e.g., at least 2%, at least 5%, at least 10%, at least 15%, at least 20%, at least 25%, at least 30%, at least 35%, at least 40%, at least 45%, at least 50%, etc.

In some embodiments, the method improves muscle function compared to muscle function in the subject before treatment. In some embodiments, the method improves skeletal muscle function compared to skeletal muscle function in the subject before treatment. Improved muscle function may include, but is not limited to, improved muscle endurance, improved muscle recovery time and improved muscle strength. In some embodiments, muscle function in the subject is increased by 1% to 90%, e.g., 1% to 70%, 1% to 50%, 1% to 30%, 5% to 50%, 5% to 30%, 10% to 30%, etc. In some embodiments, muscle function in the subject is increased by at least 1%, e.g., at least 2%, at least 5%, at least 10%, at least 15%, at least 20%, at least 25%, at least 30%, at least 35%, at least 40%, at least 45%, at least 50%, etc.

In some embodiments, the method reduces inflammation compared to inflammation in the subject before treatment. As stated previously, inflammation can present as heat, pain, redness, swelling and/or loss of function. Inflammation leads to a shift in the type of cells and/or signaling molecules present at the inflammation site. As such reduction of inflammation can present as a reduction in any of the above symptoms and/or a reduction of inflammatory cells and/or signaling molecules present at the inflammation site. Reduction of inflammation (e.g., reduction of inflammatory cells and/or signaling molecules) can be measured in a variety of methods including, but not limited to, by flow cytometry, mass spectrometry, protein array analysis, western blot analysis, enzyme-linked immunosorbent assay (ELISA), radio-immune assay (RIA), immunohistochemistry, and/or combinations thereof. In some embodiments, inflammation in the subject is reduced by 1% to 90%, e.g., 1% to 70%, 1% to 50%, 1% to 30%, 5% to 50%, 5% to 30%, 10% to 30%, etc. In some embodiments, inflammation in the subject is decreased by at least 1%, e.g., at least 2%, at least 5%, at least 10%, at least 15%, at least 20%, at least 25%, at least 30%, at least 35%, at least 40%, at least 45%, at least 50%, etc.

In some embodiments, the health of the subject is rejuvenated compared to the health of the subject before treatment. Generally, health rejuvenation refers to the practical reversal of the aging process. In other words, when the health of a subject is rejuvenated, the subject experiences anti-aging effects. Aging is the accumulation of damage to the body including, but not limited to, accumulation of damage to macromolecules, cells, tissues, and organs within the body. As such, rejuvenation is the reversal of the above-mentioned damage. In some embodiments, health rejuvenation can be quantified by measuring the increase in new and/or healthy cells. In some embodiments, health rejuvenation can be quantified a change in the distribution of types of cells. In some embodiments, health rejuvenation can be quantified by measuring the change in senescence biomarkers (i.e., biomarkers related to aging) in a cell. Changes in senescence biomarkers include, but are not limited to, an increase in a biomarker, a decrease in a biomarker, and/or a change in location of the biomarker. Senescence biomarkers include, but are not limited to, SA-β-gal, p16, p21, lamin B1, SASP components, HMGB1, yH2AX and others. Senescence biomarkers are well known in the art and have been reviewed extensively (See, e.g., Wang, et al., Front. Genet. 2018 9:247; Kudlova, et al., Int. J. Mol. Sci. 2022 12:4168; Zhang, et al., Nat. Aging 2022 2:601-615). Health rejuvenation (e.g., health rejuvenation associated cells and/or biomarkers) can be measured in a variety of methods including, but not limited to, by flow cytometry, mass spectrometry, protein array analysis, western blot analysis, enzyme-linked immunosorbent assay (ELISA), radio-immune assay (RIA), immunohistochemistry, and/or combinations thereof. In some embodiments, health rejuvenation in the subject is improved by 1% to 90%, e.g., 1% to 70%, 1% to 50%, 1% to 30%, 5% to 50%, 5% to 30%, 10% to 30%, etc. In some embodiments, health rejuvenation in the subject is improved by at least 1%, e.g., at least 2%, at least 5%, at least 10%, at least 15%, at least 20%, at least 25%, at least 30%, at least 35%, at least 40%, at least 45%, at least 50%, etc.

### Methods of Administration

Methods of administration may be carried out by any suitable means, including topical, oral, parenteral, intrapulmonary, and intranasal. Parenteral infusions include intramuscular, intravenous (bolus or slow drip), intraarterial, intraperitoneal, intrathecal or subcutaneous administration. An agent can be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also included in the disclosure, by such means as depot injections or erodible implants.

Dosage and frequency of dosing may vary depending on the half-life of the agent (e.g., an N-beta-hydroxybutyryl-amino acid) in the subject. It will be understood by one of skill in the art that such guidelines will be adjusted for the molecular weight of the active agent, the clearance from the blood, the mode of administration, and other pharmacokinetic parameters. The dosage may also be varied for localized administration, e.g., intranasal, inhalation, etc., or for systemic administration, e.g., i.m., i.p., i.v., oral, and the like.

In certain embodiments, therapeutically effective doses are administered according to a daily dosing regimen, or intermittently. In certain embodiments, therapeutically effective doses are administered daily. In some embodiments, a therapeutically effective dose is administered once. In some embodiments, a therapeutically effective dose is administered for two or more consecutive days, e.g., for two consecutive days, for three consecutive days, for four consecutive days, for five consecutive days, for six consecutive days, for seven consecutive days, for eight consecutive days, for nine consecutive days, for ten consecutive days or more, for 20 consecutive days or more, for 50 consecutive days or more, for 100 consecutive days or more. In certain embodiments, therapeutically effective doses are administered intermittently. For example, a therapeutically effective dose can be administered, one day a week, two days a week, three days a week, four days a week, or five days a week, and so forth, or, for example, every other day, every two days, every three days, once a week, once every two weeks, once every three weeks, once a month, and so forth. For example, in some embodiments, the composition is administered once every two to four weeks for an extended period of time, such as for 1, 2, 3, 4, 5, 6, 7, 8, 10, 15, 24 months, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy for one or more weekly or monthly cycles until the desired therapeutic response is achieved. The agents can be administered by any acceptable route of administration as noted above. Effective doses may likewise by administered according to any of the dosing regimens listed above.

“In combination with”, “combination therapy” and “combination products” refer, in certain embodiments, to the concurrent administration to a subject of the N-beta-hydroxybutyryl-amino acid described herein in combination with additional therapies, e.g., surgery, radiation, chemotherapy, and the like. When administered in combination, each component can be administered at the same time or sequentially in any order at different points in time. Thus, each component can be administered separately but sufficiently closely in time so as to provide the desired effect.

In some embodiments, the additional therapy comprises administering an active agent. In some embodiments, the additional therapy comprises putting the subject on a low-calorie diet. In some embodiments, the low-calorie diet comprises fewer calories than the diet of the subject before treatment. In some embodiments, the additional therapy comprises increasing the physical activity of the subject. In some embodiments, the amount of physical activity (e.g., the number of calories burned through exercise) is increased compared to the amount of physical activity of the subject before treatment. In some embodiments, the additional therapy comprises surgical intervention. The surgical invention may include, but is not limited to, bariatric surgery (e.g., sleeve gastrectomy, Roux-en-Y gastric bypass surgery, biliopancreatic diversion with duodenal switch, gastric plication, adjustable gastric band surgery, intragastric balloon surgery, implantable gastric stimulation surgery). In some embodiments, the additional therapy comprises use of a weight loss device. Weight loss devices include, but are not limited to, gastric bands, gastric balloon systems, endoscopic suturing devices, oral bite limiting devices, and stomach space-occupying devices (e.g., Plenity®).

“Concomitant administration” means administration of one or more components, such as N-beta-hydroxybutyryl-amino acids, known therapeutic agents, etc. at such time that the combination will have a therapeutic effect. Such concomitant administration may involve concurrent (i.e., at the same time), prior, or subsequent administration of components. A person of ordinary skill in the art would have no difficulty determining the appropriate timing, sequence and dosages of administration.

Compositions are typically provided in a unit dosage form, where the term “unit dosage form,” refers to physically discrete units suitable as unitary dosages for subjects (e.g., human subjects), each unit containing a predetermined quantity of active agent in an amount calculated sufficient to produce the desired effect in association with an acceptable diluent, carrier or vehicle. The specifications for the unit dosage forms of the present invention depend on the particular complex employed and the effect to be achieved, and the pharmacodynamics associated with each complex in the host.

In some embodiments, a unit dose is at least about 1 mg/kg, at least about 5 mg/kg, at least about 10 mg/kg, at least about 20 mg/kg, at least about 30 mg/kg, at least about 40 mg/kg, at least about 50 mg/kg, at least about 60 mg/kg, at least about 70 mg/kg, at least about 80 mg/kg, at least about 90 mg/kg, at least about 100 mg/kg, at least about 250 mg/kg, at least about 500 mg/kg in some embodiments the effective dose is from about 1 to 100 mg/kg.

### Methods of Supplementing

In some embodiments, the method is a method of supplementing a subject's diet. By supplementing a subject's diet, it is meant that the supplement (e.g., the N-beta-hydroxybutyryl-amino acid) is administered in addition to the subject's diet. Supplements may be orally administered. By orally administered, it is meant that the supplements are ingested (i.e., swallowed).

In certain embodiments, an effective dose (i.e., an effective amount of an N-beta-hydroxybutyryl-amino acid) is administered according to a daily dosing regimen, or intermittently. In certain embodiments, effective doses are administered daily. In some embodiments, an effective dose is administered once. In some embodiments, an effective dose is administered for two or more consecutive days, e.g., for two consecutive days, for three consecutive days, for four consecutive days, for five consecutive days, for six consecutive days, for seven consecutive days, for eight consecutive days, for nine consecutive days, for ten consecutive days or more, for 20 consecutive days or more, for 50 consecutive days or more, for 100 consecutive days or more. In certain embodiments, effective doses are administered intermittently. For example, an effective dose can be administered, one day a week, two days a week, three days a week, four days a week, or five days a week, and so forth or, for example, every other day, every two days, every three days, once a week, once every two weeks, once every three weeks, once a month, and so forth. For example, in some embodiments, the supplement is administered once every two to four weeks for an extended period of time, such as for 1, 2, 3, 4, 5, 6, 7, 8, 10, 15, 24 months, and so forth. By “twice-weekly” or “two times per week” is intended that two effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three effective doses are administered to the subject within a 7 day period, allowing for a minimum of 48 hours between doses and a maximum of 72 hours between doses. Supplement compositions may be provided in any of the unit doses listed above.

### Compositions

Aspects of the present disclosure include compositions comprising an N-beta-hydroxybutyryl-amino acid and an acceptable excipient.

A “composition” refers to a formulation of a compound and a medium generally accepted in the art for the delivery of the biologically active compound to a mammal, e.g., humans. Such a medium can include an acceptable delivery vehicle, carrier, diluent, or excipient. Compositions include “pharmaceutically acceptable compositions” wherein delivery vehicles, carriers, diluents and/or excipients are pharmaceutically acceptable.

While it may be possible for the compounds of the subject invention to be administered as the raw chemical, it is also possible to present them as a composition. Accordingly, provided herein are compositions which comprise one or more of certain compounds disclosed herein, or one or more acceptable salts, esters, prodrugs, amides, or solvates thereof, together with one or more acceptable carriers (e.g., excipients) thereof and optionally one or more other therapeutic ingredients.

Acceptable delivery vehicles and other therapeutic ingredients may include one or more excipients and/or one or more vehicles. The excipient(s) or vehicles(s) must be “acceptable” in the sense of being compatible with the other ingredients of the formulation and not deleterious to the recipient thereof. Proper formulation is dependent upon the route of administration chosen. Any of the well-known techniques, vehicles, carriers, and excipients may be used as suitable and as understood in the art; e.g., in Remington's Pharmaceutical Sciences. The compositions disclosed herein may be manufactured in any manner known in the art, e.g., by means of conventional mixing, dissolving, granulating, dragee-making, levigating, emulsifying, encapsulating, entrapping or compression processes.

As noted above, an agent, such as N-beta-hydroxybutyryl-amino acid, can be formulated with an acceptable excipient (one or more organic or inorganic ingredients, natural or synthetic, with which a subject agent is combined to facilitate its application). A suitable delivery vehicle includes sterile saline although other aqueous and non-aqueous isotonic sterile solutions and sterile suspensions known to be acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

An agent, such as an N-beta-hydroxybutyryl-amino acid, can be administered as a composition comprising an acceptable excipient. The preferred form depends on the intended mode of administration and therapeutic application. The compositions can also include, depending on the formulation desired, pharmaceutically-acceptable and/or non-toxic carriers or diluents, which are defined as vehicles commonly used to formulate compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the composition or formulation may also include other carriers, adjuvants, or nontoxic, nontherapeutic, nonimmunogenic stabilizers and the like.

Compounds useful for co-administration with the active agents, such as N-beta-hydroxybutyryl-amino acids, of the invention can be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

Compounds may also be purchased. Such “commercially available” compounds may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

In dosage forms, the active agents and/or other compounds may be administered in the form of their acceptable salts, or they may also be used alone or in appropriate association, as well as in combination with other active compounds. The agents may be combined, as previously described, to provide a cocktail of activities. The following methods and excipients are exemplary and are not to be construed as limiting the invention.

The acceptable excipients, such as vehicles, adjuvants, carriers or diluents, are commercially available. Moreover, acceptable auxiliary substances, such as pH adjusting and buffering agents, tonicity adjusting agents, stabilizers, wetting agents and the like, are commercially available. Any compound useful in the methods and compositions of the invention can be provided as an acceptable base addition salt. “Acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier (e.g., excipient) may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. The nature of the carrier can be either soluble or insoluble for purposes of the invention.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249:1527, 1990 and Hanes, Advanced Drug Delivery Reviews 28:97-119, 1997. The agents of this invention can be administered in the form of a depot injection or implant preparation which can be formulated in such a manner as to permit a sustained or pulsatile release of the active ingredient. The compositions are generally formulated as sterile, substantially isotonic and in full compliance with all Good Manufacturing Practice (GMP) regulations of the U.S. Food and Drug Administration.

Compositions can be prepared as formulations to be taken orally. The various embodiments and N-beta-hydroxybutyryl-amino acid compositions of the instant invention may be prepared for oral administration according to any of the methods, techniques, and/or delivery vehicles described herein. Further, one having skill in the art will appreciate that the N-beta-hydroxybutyryl-amino acid molecule compositions of the instant invention may be modified or integrated into a system or delivery vehicle that is not disclosed herein, yet is well known in the art and compatible for use in oral delivery of amino acid molecules.

Oral dosage forms or unit doses compatible for use with the N-beta-hydroxybutyryl-amino acids of the present invention may include a mixture of N-beta-hydroxybutyryl-amino acids, and other components or excipients, as well as other non-reusable materials that may be considered either as an ingredient or packaging. Oral compositions may include at least one of a liquid, a solid, and a semi-solid dosage forms. In some embodiments, an oral dosage form is provided comprising an effective amount of a peptide molecule described herein, wherein the dosage form comprises at least one of a pill, a tablet, a capsule, a gel, a paste, a drink, and a syrup. In some instances, an oral dosage form is provided that is designed and configured to achieve delayed release of the N-beta-hydroxybutyryl-amino acid in the small intestine of the subject.

### Supplements

A “supplement” is a composition comprising any of the N-beta-hydroxybutyryl-amino acids described herein. In some embodiments, the N-beta-hydroxybutyryl-amino acid comprises an amino acid with a hydrophobic side chain. In some embodiments, the N-beta-hydroxybutyryl-amino acid is N-beta-hydroxybutyryl-phenylalanine (BHB-Phe), N-beta-hydroxybutyryl-leucine (BHB-Leu), N-beta-hydroxybutyryl-isoleucine (BHB-Ile), N-beta-hydroxybutyryl-valine (BHB-Val), or N-beta-hydroxybutyryl-methionine (BHB-Met). The terms “dietary supplement” or “supplement” or “supplement composition” may be used interchangeably herein to describe compositions administered to a subject to maintain and/or increase the health of the subject. Dietary supplements (e.g., supplements comprising N-beta-hydroxybutyryl-amino acids), are intended to add to or supplement the subject's diet.

As reviewed above, in some embodiments, supplements comprising an N-beta-hydroxybutyryl-amino acid of the present invention may reduce food intake, reduce body weight, increase glucose clearance, reduce adipose tissue mass, increase muscle mass, reduce inflammation and/or rejuvenate health. As reviewed above, in some embodiments, the supplement comprising the N-beta-hydroxybutyryl-amino acid exhibits physiological activity. In some embodiments, the physiological activity is hypophagic activity.

Supplements may be formulated for oral administration. Supplements may be orally administered in various forms including, but not limited to, tablets, capsules, soft gels, gel caps, powders, bars, gummies, pastes, liquids, shakes, bars, confectionaries, and supplemented foods. Supplements may be administered according to any dosage and/or dosing regimen for compositions described herein. In some embodiments, the supplements may be administered for two or more consecutive days.

Supplements may comprise “other ingredients” besides the “dietary ingredients” (i.e., N-beta-hydroxybutyryl-amino acids). “Other ingredients” include, but are not limited to, substances and/or excipients, such as fillers, binders, preservatives, sweeteners, colorings and flavorings. “Other ingredients” that are suitable for supplements comprising N-beta-hydroxybutyryl-amino acids are any ingredients that are non-toxic and do not interfere with and/or compromise the intended effect of the supplement in a subject.

Fillers include, but are not limited to, magnesium stearate, silicon dioxide, titanium dioxide, starch, cellulose (e.g., microcrystalline cellulose), stearic acid, simethicone, vegetable gum, talc, mannitol, lactose and propylene glycol. Binders include, but are not limited to, gum arabic, acacia gum, gelatin. Preservatives include, but are not limited to, sodium benzoate, methyl parabens, propyl parabens, sodium nitrite, sulfur dioxide, sodium sorbate, potassium sorbate, citric acid, malic acid and tartaric acid. Sweeteners may be natural sweeteners and/or artificial sweeteners. Sweeteners include, but are not limited to fructose, glucose, sucrose and low-calorie sweeteners (e.g., sucralose). Colorings may be natural colorings and/or artificial colorings. Flavorings may be natural flavorings and/or artificial flavorings.

In some embodiments, the supplements are formulated so the percentage of the dietary ingredient (i.e., N-beta-hydroxybutyryl-amino acid) is about 1% to about 99% by weight, including, about 1% to about 90% by weight, about 5% to about 95% by weight, about 10% to about 90% by weight, about 20% to about 90% by weight, about 30% to about 90% by weight, about 50% to about 90% by weight, about 50% to about 80% by weight, about 60% to about 80% by weight, or about 70% to about 80% by weight, etc.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1—CNDP2 Catalyzes BHB-Ylation of Amino Acids In Vitro and in Cells

It has previously been demonstrated that CNDP2 can catalyze the condensation of lactate with phenylalanine and other amino acids to generate Lac-Phe and other N-lactoyl amino acids in vitro (Li et al., Nature 2022 606:785-790). One representative member, Lac-Phe (N-lactoyl phenylalanine), is an exercise-inducible metabolite that suppresses food intake. Genetic ablation of CNDP2 dramatically reduces Lac-Phe levels and increases food intake and body weight after exercise training. CNDP2 is highly expressed in immune cells and epithelial cells of the gut and kidney, rather than classical tissues associated with lactate metabolism such as muscle or liver (Schaum, et al., Nature 2018 562:367-372). These observations demonstrate that the enzymatic pathways of lactate metabolism extend beyond glycolysis to include CNDP2+ cells and lactate-derived signaling metabolites.

Despite profound physiologic differences in production and regulation, BHB and lactate exhibit a surprising degree of chemical similarity. First, BHB and lactate are structurally similar, being both hydroxycarboxylic acids that differ by only a single methylene (C3 for lactate vs. C4 for BHB). Second, BHB and lactate are both transport substrates for the monocarboxylate transporters (MCTs), demonstrating that their structural similarity also translates to a functional similarity in molecular recognition, at least with respect to the MCT active site.

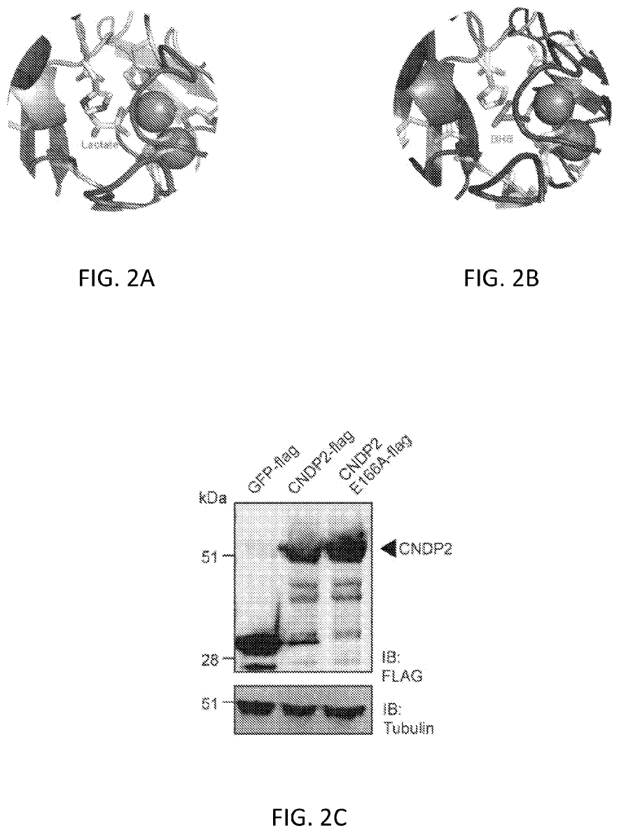

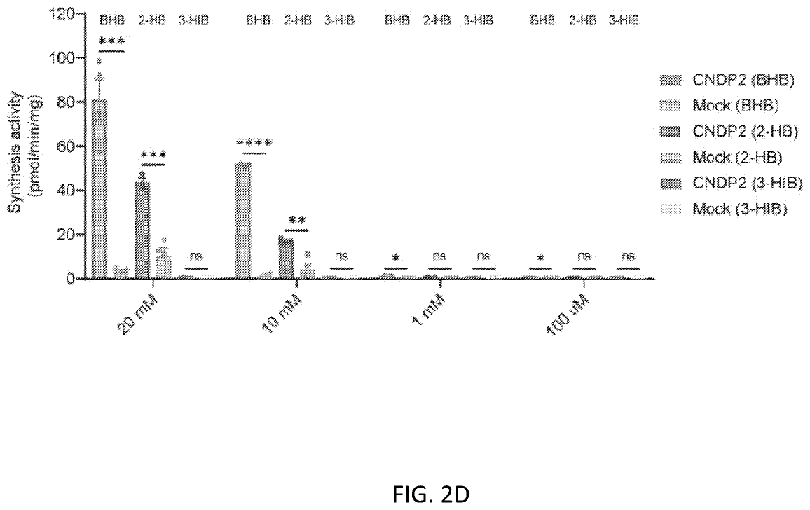

Based on the structural similarity between lactate and BHB (FIG. 1A), it was hypothesized that CNDP2 might exhibit enzyme active site promiscuity and, in addition to lactate (Li et al., Nature 2022 606:785-790), also accept BHB as a condensation substrate with phenylalanine, and possibly other amino acids as well (FIG. 1B). Further supporting this hypothesis, a molecular docking simulation of BHB and lactate into the CNDP2 active site revealed near equivalent modeled binding affinities (lactate,−4.8 kcal/mol; BHB,−5.1 kcal/mol, FIGS. 2A-2B and see Methods). To experimentally determine whether CNDP2 can catalyze the BHB-ylation of free amino acids in vitro, BHB and phenylalanine were incubated with cell lysates from HEK293T cells that were transiently transfected with flag-tagged CNDP2 or GFP control. The formation of the BHB-ylation product Keto-Phe (N-betahydroxybutyryl phenylalanine) was monitored by liquid chromatography-mass spectrometry (LC-MS). As shown in FIG. 1C, CNDP2-transfected cell lysates exhibited >140-fold greater phenylalanine BHB-ylation activity compared to GFP-transfected cell lysates. Importantly, a CNDP2-E166A mutant in which an active site residue required for enzyme activity is mutated to alanine completely abolished the synthetase activity (FIG. 1C and FIG. 2C), demonstrating that the BHB-ylation activity is entirely encoded within the CNDP2 polypeptide sequence. A direct comparison of CNDP2 enzyme activities revealed similar rates of amino acid BHB-ylation and N-lactoylation (FIG. 1D). By contrast, CNDP2 was unable to accept longer (e.g., octanoate, C8) or shorter (e.g., acetate, C2) acids as substrates (FIG. 1D). Lastly, the amino acid selectivity for the BHB-ylation reaction was examined. As shown in FIG. 1E, CNDP2 catalyzed BHB-ylation of several amino acids with hydrophobic side chains, including phenylalanine, leucine/isoleucine, valine, and to a lesser extent, methionine. Little activity was observed when other amino acids were used as substrates.

To determine if CNDP2 could catalyze BHB-ylation of amino acids in a more complex cellular environment, an in situ, live cell enzyme activity assay was performed. HEK293T cells were transfected with plasmids encoding CNDP2 or GFP and then treated with BHB (5 mM). Extracellular metabolites were harvested with acetonitrile/methanol extraction of the conditioned media. Multiple keto amino acids in media were significantly increased after BHB treatment in CNDP2-transfected cells (FIG. 1F). Notably, the same subset of hydrophobic keto amino acids produced by CNDP2 in vitro, including Keto-Phe, Keto-(iso) Leu, and Keto-Val, also exhibited robust production in CNDP2-transfected live cells. By contrast, little keto amino acids were produced in control GFP-transfected cells (FIG. 1F). Lastly, the intracellular versus extracellular distribution of keto amino acids was examined. Similar to what was previously reported for N-lactoyl amino acids, keto amino acids were predominantly found in the media, establishing that these metabolites are also robustly effluxed from cells (FIG. 1G). This data demonstrates that CNDP2 exhibits multifunctional biosynthetic activity and can catalyze the BHB-ylation of free amino acids in vitro and the production of keto amino acids in cells.

To quantitatively compare the kinetics of CNDP2-dependent BHB-ylation and N-lactoylation, purified recombinant mouse CNDP2-flag protein was generated for in vitro kinetic assays with increasing concentrations of either BHB or lactate. The resulting kinetic data, which were fit to Michaelis-Menten kinetics, revealed that BHB is in fact a higher affinity substrate for the CNDP2 active site than lactate (Km for BHB=8.8 mM; Km for lactate=33.4 mM, FIGS. 1H-11). By contrast, CNDP2-dependent Lac-Phe synthesis is faster than that of BHB-Phe synthesis under conditions of saturating substrate concentrations (Vmax for lactate=62.7 nM/min/mg, Vmax for BHB=3.5 nM/min/mg, FIGS. 1H-11).

Lastly, the amino acid condensation activity of CNDP2 was compared with other structural isomers of BHB, including 3-hydroxisobutyric acid (3-HIB) and 2-hydroxybutyrate (2-HB) (Nilsen, et al., Diabetes 2020 69:1903-1916; Gall, et al., PloS One 2010 5: e10883). CNDP2-dependent condensation activity using 3-HIB as a substrate was not detected (FIG. 2D). Modest CNDP2-dependent production of 2-HB-Phe was observed, but at a rate only <50% that of BHB-Phe, and only at 2-HB concentrations of 10 mM or greater (FIG. 2D). In conclusion, BHB is the preferred substrate for CNDP2 amongst multiple isomeric C4-hydroxycarboxylic acid substrates.

### Example 2—CNDP2 is the Principal Enzyme Responsible for Amino Acid BHB-Ylation in Mouse tissues

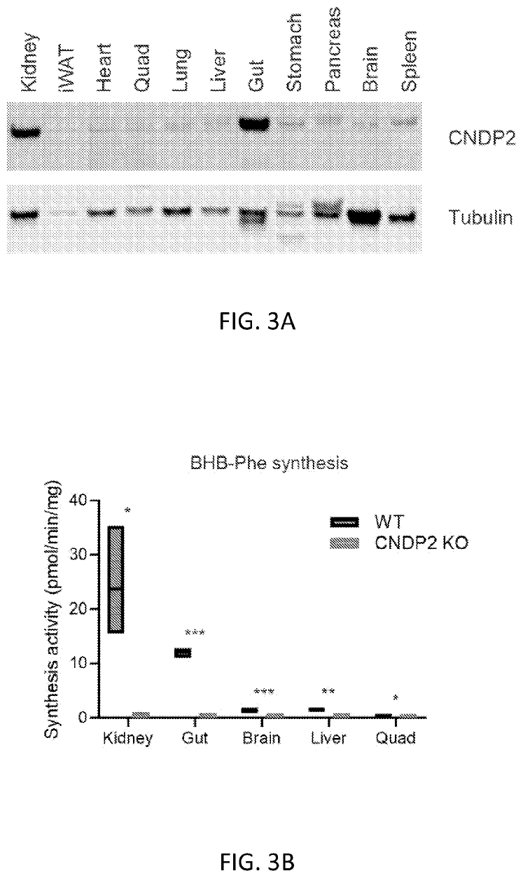

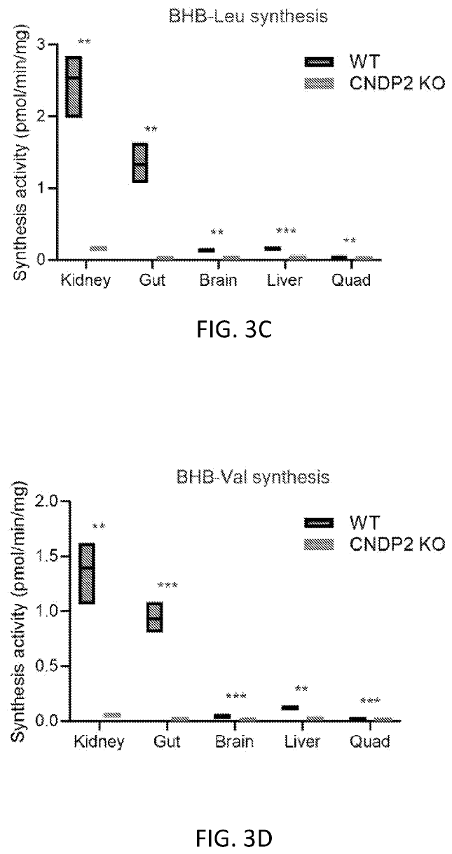

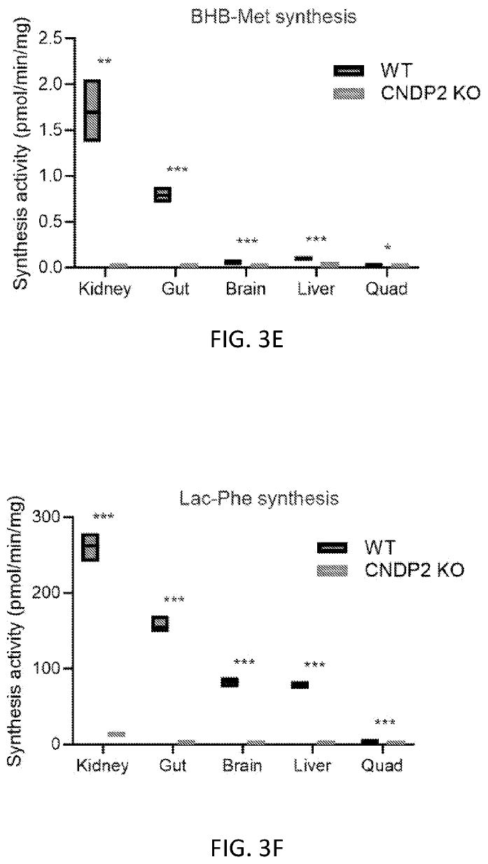

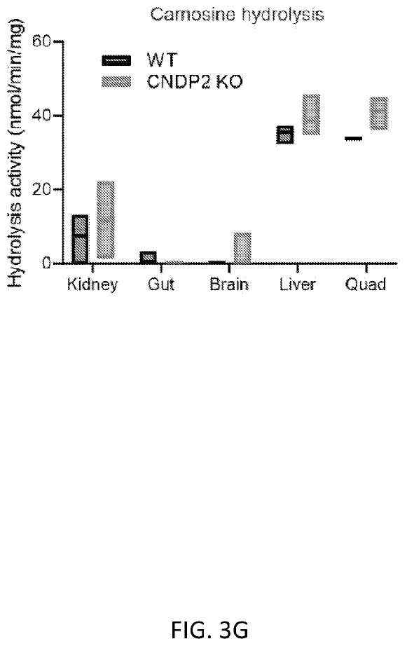

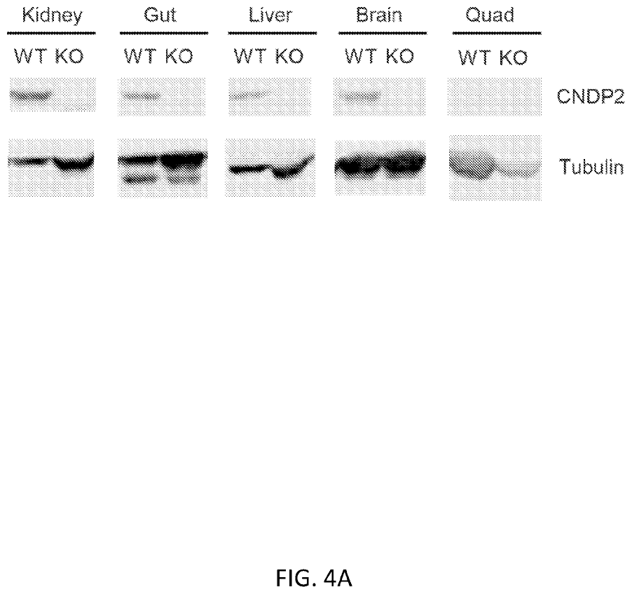

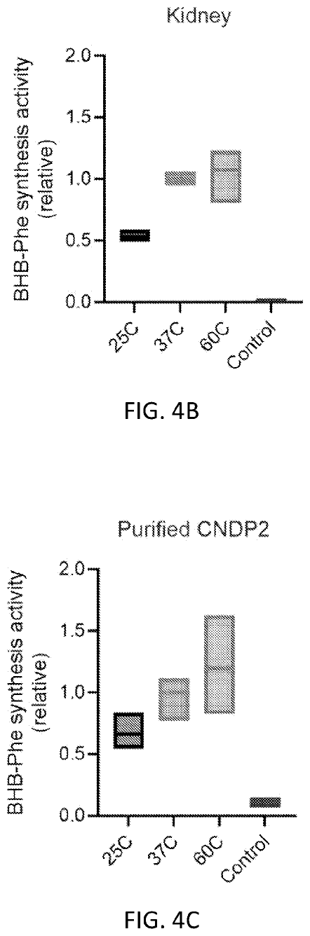

To determine if endogenous CNDP2 catalyzes amino acid BHB-ylation in mouse tissues, the tissue expression of CNDP2 was examined by Western blot using an anti-CNDP2 antibody. The specificity of this antibody was confirmed using tissues from CNDP2-KO mice (FIG. 4A). CNDP2 was widely expressed, with the highest levels in kidney and gut, and lower levels in many of the other tissues examined (FIG. 3A). Next, in vitro BHB-Phe synthesis activity was performed using crude total lysates of kidney, gut, brain, liver, or quadriceps tissues from WT (see Methods). These tissues were selected because of their wide range of CNDP2 protein expression. As shown in FIG. 3B, the highest BHB-Phe synthesis activity was observed in kidney and gut, while brain, liver, and quadriceps exhibited lower, but detectable BHB-Phe synthesis activity. This pattern of BHB-Phe synthesis across tissues largely paralleled the protein expression of CNDP2 in these same tissues. In addition, the temperature dependence of the renal BHB-Phe synthesis activity also paralleled that of recombinant CNDP2 protein (FIGS. 4B-4C). Therefore, the tissue expression pattern and temperature profile of CNDP2 protein both correlate with the tissue BHB-Phe synthesis activity.

Tissues from CNDP2-KO mice were used to determine the contribution of CNDP2 to the tissue BHB-Phe synthesis activity. As shown in FIG. 3B, both kidney and gut BHB-Phe synthesis activity was largely abolished (>95%) in tissues from CNDP2-KO mice. The smaller BHB-Phe synthesis activity in other tissues was also greatly diminished (>85% reduced in brain, >75% reduced in liver, and >60% reduced in quadriceps). Using leucine, valine, and methionine as substrates, a similar pattern of BHB-Leu, BHB-Val, and BHB-Met synthesis across WT and CNDP2-KO tissues was observed (FIGS. 3C-3E): kidney and gut tissues from WT mice both exhibited the highest BHB-amino acid synthesis activity, and this activity was largely abolished in tissues from CNDP2-KO mice. In additional control experiments, tissue Lac-Phe synthesis activity was assayed, which again exhibited a similar pattern and CNDP2-dependence to that of BHB-amino acid synthesis (FIG. 3F). By contrast, carnosine hydrolysis across tissues exhibited a distinct pattern with highest activity in liver and quadriceps and little activity in the kidney, gut, and brain; in addition, the tissue carnosinase activity was not sensitive to genetic ablation of CNDP2 (FIG. 3G). CNDP2 is therefore not a major carnosinase in vivo, despite its previously annotated in vitro activity. In conclusion, CNDP2 is the principal enzyme responsible for BHB-amino acid synthesis activity in mouse tissues.

### Example 3—Detection and Dynamic Regulation of Keto Amino Acids in Mouse Plasma

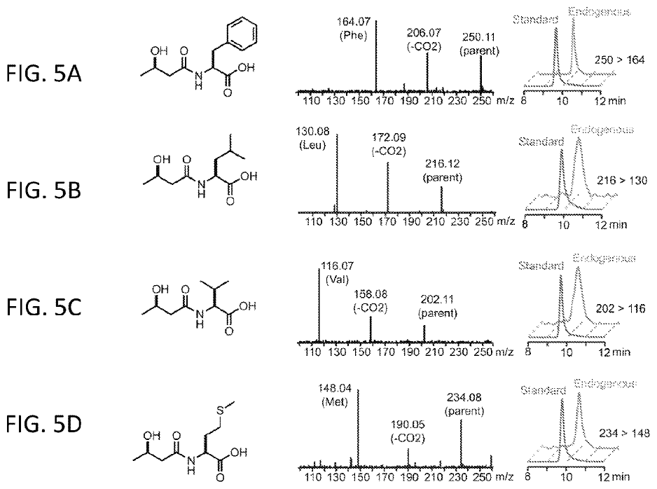

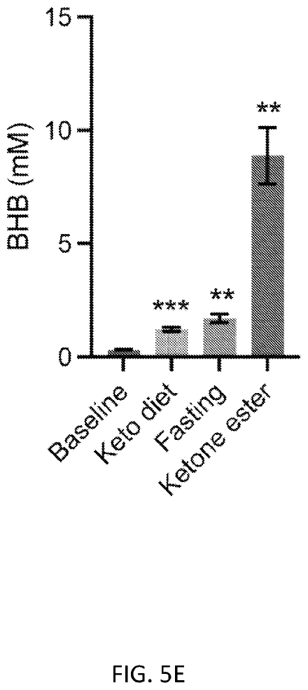

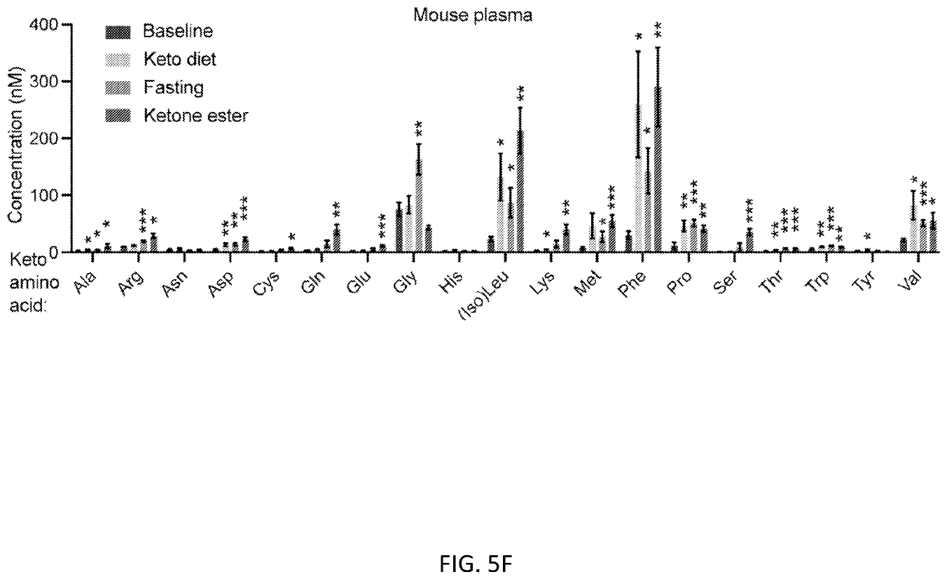

To determine whether keto amino acids are in fact endogenous mouse metabolites, authentic standards for Keto-Phe, Keto-Val, Keto-Leu, and Keto-Met were synthesized by classical amide coupling between BHB and the corresponding amino acid (FIGS. 5A-5D and see Methods). Fragmentation of these authentic standards revealed a major daughter ion corresponding to the free amino acid (m/z=164, phenylalanine; m/z=116, valine; m/z=130, leucine; m/z=148, methionine) and a second, smaller daughter ion corresponding to decarboxylation of the parent mass (m/z=206, Keto-Phe; m/z=158, Keto-Val; m/z=172, Keto-Leu; m/z=190, Keto-Met, FIGS. 5A-5D). This data demonstrated that keto amino acids exhibit a predicable MS/MS pattern characterized by either loss of the amino acid head group or loss of carbon dioxide. Next, a targeted multiple reaction monitoring (MRM) method on a high-performance liquid chromatography coupled to triple quadrupole mass spectrometry (QQQ-LC/MS) was developed to monitor the parent to amino acid transitions for each of the four keto amino acids in mouse plasma. An endogenous peak at the appropriate parent-to-daughter transition that eluted at an identical time with the authentic keto amino acid standards was observed for each of the four keto amino acids (FIGS. 5A-5D). Therefore, keto amino acids are endogenous metabolites in mouse plasma.

Because of their biosynthetic origin from BHB, circulating keto amino acids would be predicted to rise with increasing BHB levels, such as those achieved by nutritional or physiologic ketosis. Levels of keto amino acids in mouse blood plasma were therefore measured after one week of ketogenic diet (Research Diets D21021808), a 24 h fast, or oral administration of a ketone ester drink (3 g/kg of body weight). For those keto amino acids for which an authentic standard had not been synthesized, a parent to amino acid transition was extrapolated based on the calculated m/z of the parent and corresponding amino acid daughter ion. It was confirmed that plasma BHB levels were elevated by each of these conditions (FIG. 5E). As expected, many keto amino acids were also detectable and elevated under all three conditions that depended on the specific perturbation and specific keto amino acid species (FIG. 5F). The two physiologic ketosis stimuli, ketogenic diet and fasting, consistently produced robust 2-10-fold elevations across the four keto amino acids previously identified to be produced by CNDP2 in vitro (Keto-(iso) Leu, Met, Phe, and Val), as well as several others that had not been previously observed to be directly regulated by CNDP2 in vitro (e.g., Keto-Ala and Keto-Pro). Pharmacological ketone ester administration produced the greatest induction in keto amino acids levels, which paralleled the high BHB levels achieved with this intervention (FIGS. 5E, 5F). Therefore, keto amino acids are ketosis-inducible circulating metabolites in mouse plasma.

Keto amino acids were robustly detectable in our mass spectrometry analysis, but not annotated in prior metabolic studies. First, the enzymological studies of CNDP2 provided herein, and the close chemical parallels between lactate and BHB, provided a compelling and plausible biochemical hypothesis for the biosynthetic origins of keto amino acids. Second, while authentic samples of keto amino acids were not commercially available, synthesis of these standards enabled confirmation of retention time and fragmentation. This strategy for detecting keto amino acids suggest that metabolome space might be more generally annotated by combining authentic metabolite standards with hypotheses about the chemical similarity of substrates and promiscuity of biochemical reactions.

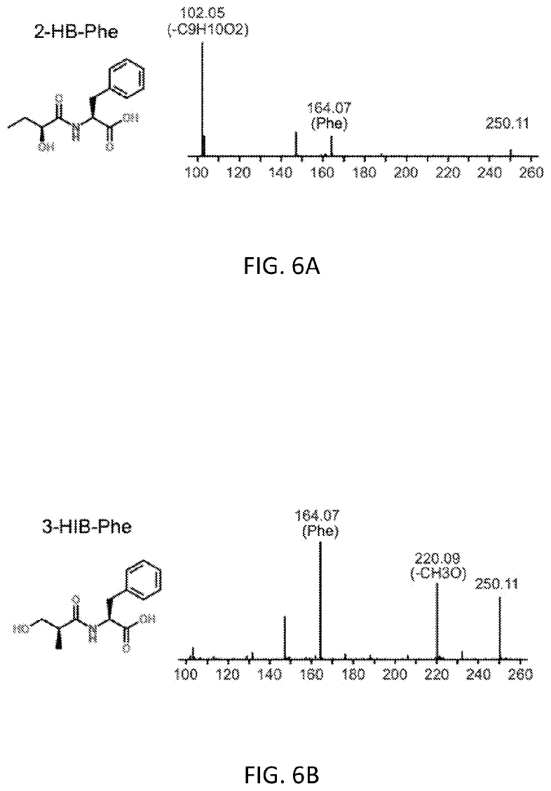

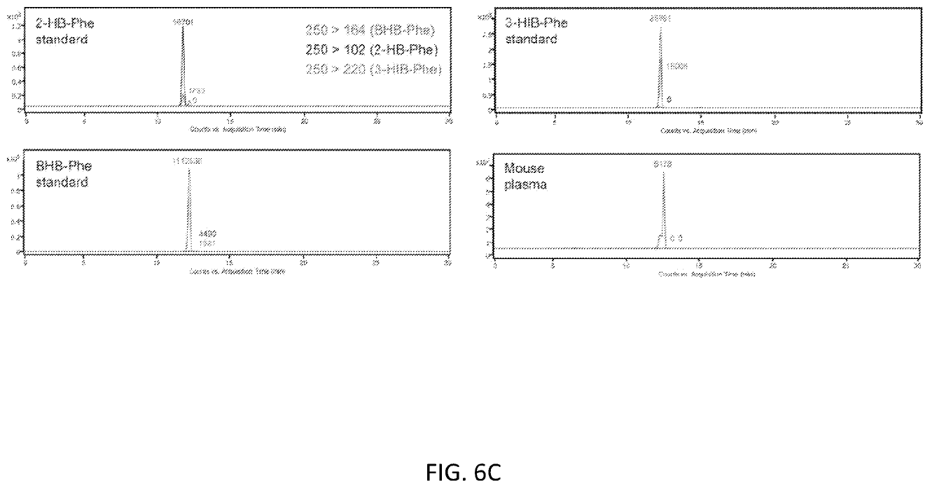

To exclude the possibility that out MRM method may also be detecting isobaric 2-hydroxybutyrate (2-HB)-and 3-hydroxyisobutyrate (3-HIB)-phenylalanine isomers, authentic standards of 2-HB-Phe and 3-HB-Phe were synthesized and MRM methods were developed that could distinguish between each of the three molecules. While 2-HB-Phe and 3-HIB-Phe yielded daughter ions corresponding to phenylalanine, unique transitions were also identified (2-HB-Phe: 260>102, fragmentation at N—Cα; 3-HIB-Phe: 250>220, loss of CH₃O, FIGS. 6A-6B). For BHB-Phe, >99% of the total signal was detected using the 250>164 transition, 2-HB-Phe was detected using both 260>102 and 250>164 (in a 6:1 ratio), and 3-HIB-Phe was detected using both 250>220 and 250>164 (in a 1.6:1 ratio) (FIG. 6C). The signal from the endogenous peak was found to be comprised >99% of the 260>164 transition (FIG. 6C). Therefore, the endogenous signal is BHB-Phe; in addition, 2-HB-Phe and 3-HIB-Phe are not endogenous metabolites.

### Example 4—Biochemical and Cellular Organization of Amino Acid BHB-ylation, Lactate, Ketolysis Pathways In Vivo

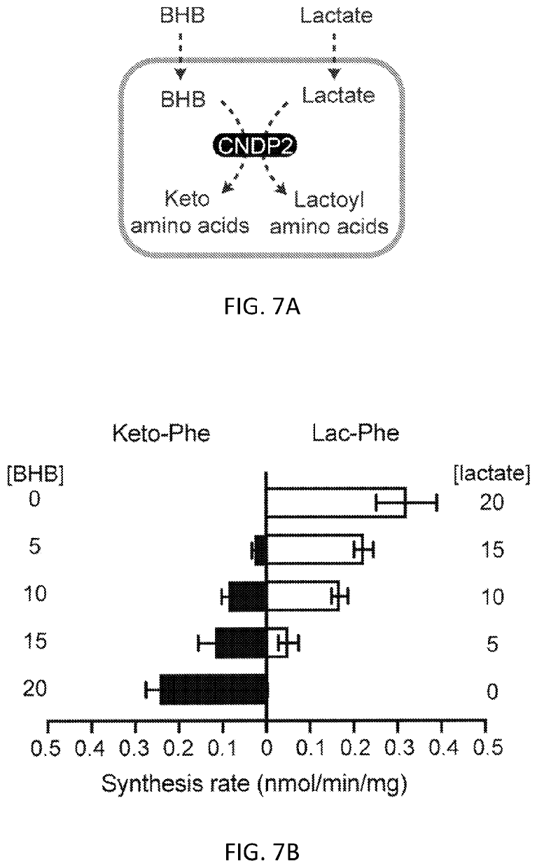

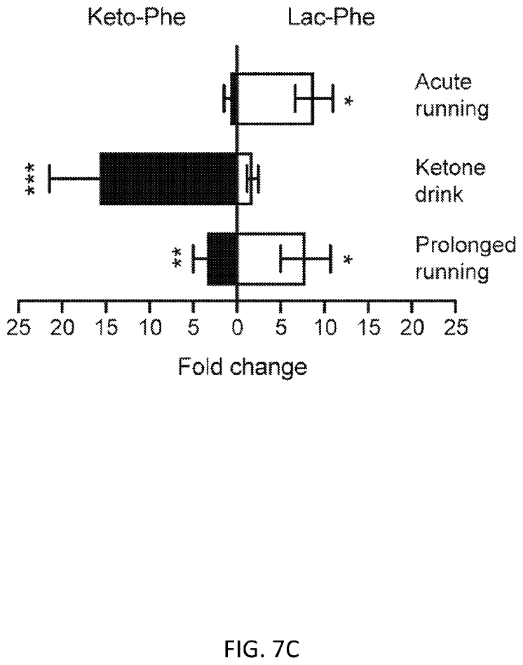

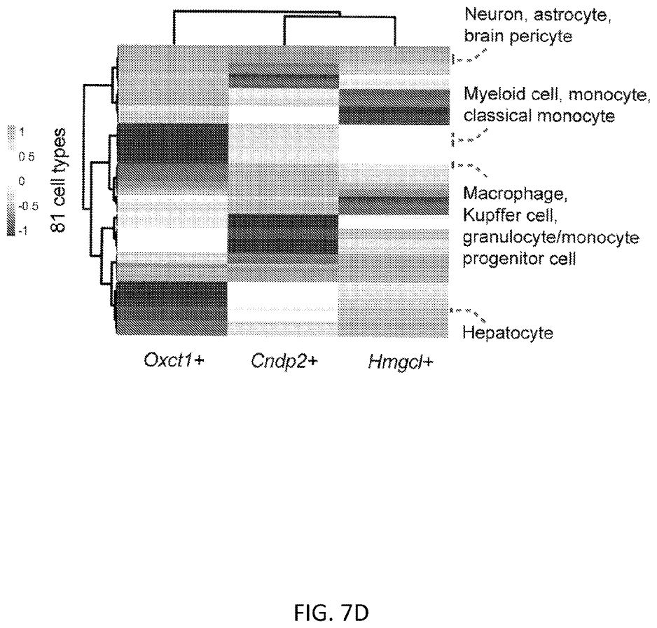

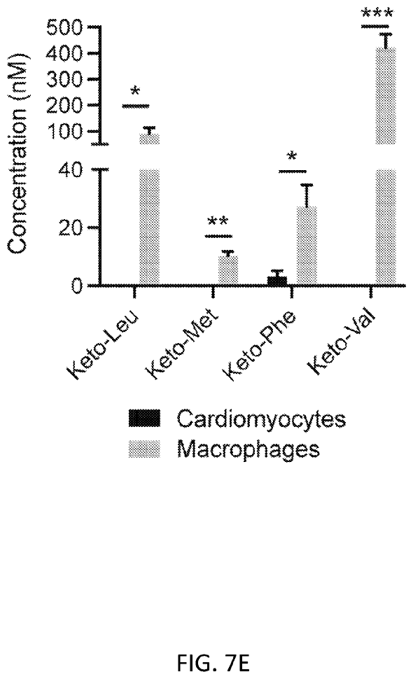

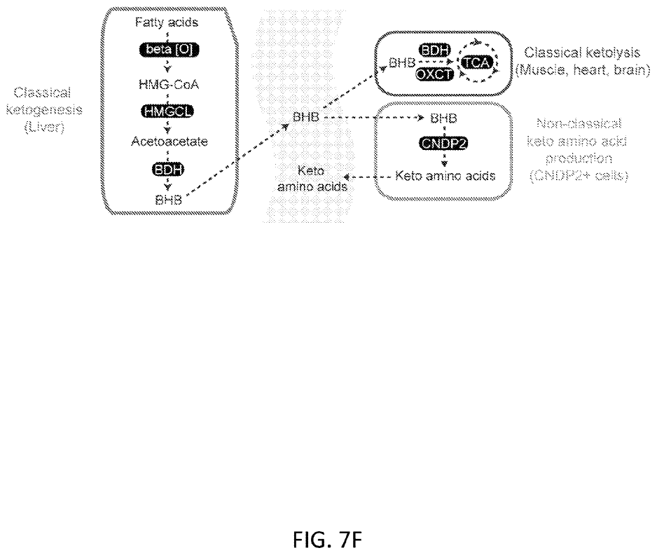

First, it was investigated how non-classical CNDP2-dependent amino acid BHB-ylation intersects with other reported CNDP2 biosynthetic activities (e.g., Lac-Phe biosynthesis) and the other classical pathways of BHB metabolism (e.g., biosynthesis and catabolismo understand the biochemical interplay between BHB and lactate as CNDP2 substrates (FIG. 7A), in vitro CNDP2-dependent lactoylation or BHB-ylation activity with either lactate alone, BHB alone, or some combination of the two substrates at a defined stoichiometry were measured. When lactate and phenylalanine were provided to CNDP2 as the only two substrates, exclusive production of Lac-Phe was observed (FIG. 7B, right). Similarly, when BHB and phenylalanine were provided as the only two substrates, Keto-Phe was the exclusive product formed (FIG. 7B, left). A combination of lactate and BHB at varying concentrations resulted in simultaneous production of both Lac-Phe and Keto-Phe in ratios that paralleled the corresponding amount of starting material (either lactate or BHB, respectively) provided. To determine whether such substrate-driven mass action-based enzymology might also occur in vivo, Lac-Phe and Keto-Phe were measured in mice after a short acute bout of treadmill running exercise or after administration of a ketone ester drink. These two perturbations were selected as distinct physiologic stimuli that would result in selective elevation of either lactate or BHB, respectively. As shown in FIG. 7C, ketone ester administration resulted in >10-fold elevation of Keto-Phe without affecting Lac-Phe levels. Conversely, a short bout of intense treadmill running (15 min) resulted in dramatic and selective elevation of Lac-Phe by 8-fold, whereas Keto-Phe was unchanged. Lastly, upon strenuous and prolonged exercise, when both lactate and BHB are elevated, robust co-induction of Lac-Phe and Keto-Phe was observed (FIG. 7C). In conclusion, CNDP2 is a multi-functional enzyme that drives the production of several N-acyl amino acid conjugates in a manner dependent on substrate availability in vitro and in vivo.

Having established a dual substrate model for CNDP2, the relationship of this BHB-dependent enzymatic pathway with the other primary pathways of BHB metabolism was investigated. That Cndp2 mRNA has been previously reported to be highly expressed in macrophages (Schaum, et al., Nature 2018 562:367-372) and other cell types not classically associated with BHB production (Goldberg, et al., J. Biol. Chem. 2023 299) or catabolism (Puchalska, et al., Cell Metab. 2019 29:383-398.e7) suggested a potential cellular segregation of enzymatic BHB-ylation and classical BHB metabolic pathways. To systematically probe this intuition across all cell types of the mouse, public single-cell data from Tabula Muris was re-analyzed (Schaum, et al., Nature 2018 562:367-372). Cells capable of BHB production were identified by the expression of the BHB biosynthetic enzyme Hmgcl+; BHB catabolism by expression of Oxct1+, an enzyme that encodes SCOT (succinyl-CoA: 3-ketoacid CoA transferase); and amino acid BHB-ylation by expression of Cndp2. Notably, these markers defined three cell populations that were largely non-overlapping (FIG. 7D). For instance, amongst the highest 10% of Cndp2+expressing cells, only 1 out of 8 (13%) were also within the top 10% of Oxct1+ cells. These observations suggested that the two BHB-consuming pathways, BHB-ylation and BHB catabolismare functional segregation by cell type. To empirically test this prediction, primary cardiomyocytes (a representative cell type that catabolizes BHB) or RAW264 macrophages (a representative cell type that catalyzes BHB-ylation) were treated with BHB and measured production of keto amino acids. Only macrophages efficiently produced multiple keto amino acids, whereas primary cardiomyocytes only produced a minor amount of Keto-Phe but not any of the other keto amino acids (FIG. 7E). This data demonstrates that the classical pathways of BHB metabolism, and the non-classical pathway of keto amino acid biosynthesis are spatially and anatomically segregated in a cell type-specific manner (FIG. 7F)

### Example 5—Genetic and Enzymatic Regulation of Keto Amino Acids

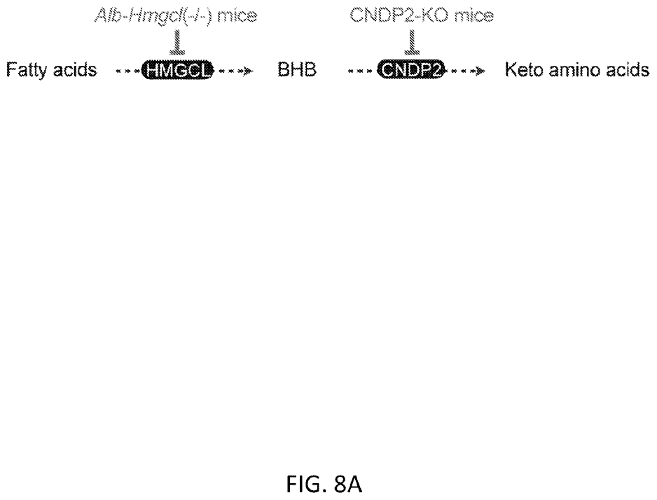

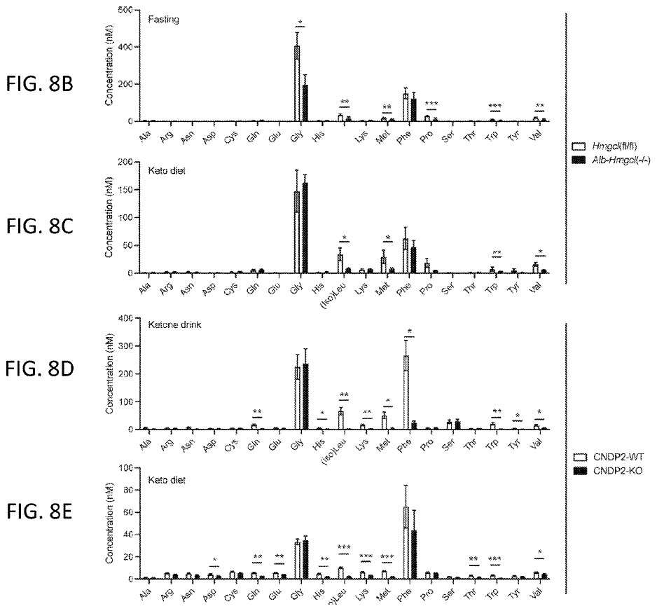

To understand the genetic and enzymatic pathways that regulate circulating keto amino acids in vivo, two genetic approaches were used to manipulate candidate enzymes involved in the metabolism of keto amino acids. First, the effects of liver-specific deletion of HMGCL, a critical upstream enzyme involved in ketogenesis, were examined (FIG. 8A). Plasma was obtained from liver-specific knockouts of HMGCL (Alb-Hmgcl (−/−) mice) which were previously generated by crossing Albumin-cre driver mice with Hmgcl floxed mice. Alb-Hmgcl (−/−) mice had previously been reported to have reductions in circulating BHB levels after fasting (24 h) or ketogenic diet (2 days) (Goldberg, et al., J. Biol. Chem. 2023 299). Upon re-analysis of their blood plasma using our targeted keto amino acid LC-MS method, multiple species were identified, including Keto-(iso) Leu, Keto-Met, and Keto-Val, that were consistently reduced by 50-80% in plasma from Alb-Hmgcl (−/−) after both 24 h fasting and 2 days of ketogenic diet (FIGS. 8B-8C). Several other keto amino acids were reduced only following either fasting or ketogenic diet, potentially reflecting the different temporal dynamics of these two physiologic perturbations.

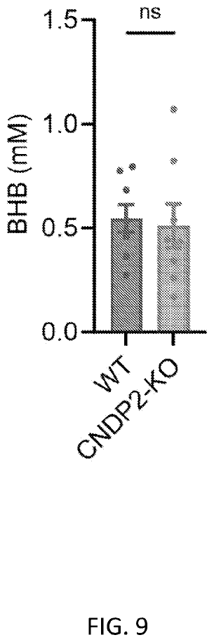

Next, the role of CNDP2 as the key and most proximal upstream biosynthetic enzyme of keto amino acids was investigated in vivo (FIG. 8A). To do this, keto amino acids were measured in blood plasma from CNDP2-KO mice after a ketone ester drink challenge or after one week of ketogenic diet (Research Diets, D06040601). In both perturbations, levels of plasma BHB itself were unchanged between WT and CNDP2-KO mice (FIG. 9), demonstrating that CNDP2-catalyzed BHB-ylation of amino acids does not directly affect total pools of circulating BHB. CNDP2-KO mice also exhibited robust depletion of many keto amino acids compared to wild type mice (FIGS. 8D-8E). This set of CNDP2-regulated keto amino acids partially overlapped with the depletion of keto amino acids observed in Alb-Hmgcl (−/−) mice. Notably, the levels of other keto amino acids, such as Keto-Pro, which were reduced in Alb-Hmgcl (−/−) mice and unaltered in the CNDP2-KO mice, suggesting the presence of potentially distinct enzymes that preferentially catalyze BHB-ylation in an amino acid-dependent manner. Lastly, it was observed that many more keto amino acids depleted after one week of ketogenic diet in CNDP2-KO mice versus wild-type controls, which may reflect the longer duration of this challenge and potential secondary biochemical effects such as acyltransferase reactions. Together, this data establishes the genetic requirement for two upstream enzymes, HMGCL and CNDP2, in the regulation of total circulating keto amino acids levels.

### Example 6—Keto Amino Acids Directly Bind to Proteins in Tissue Lysates In Vitro

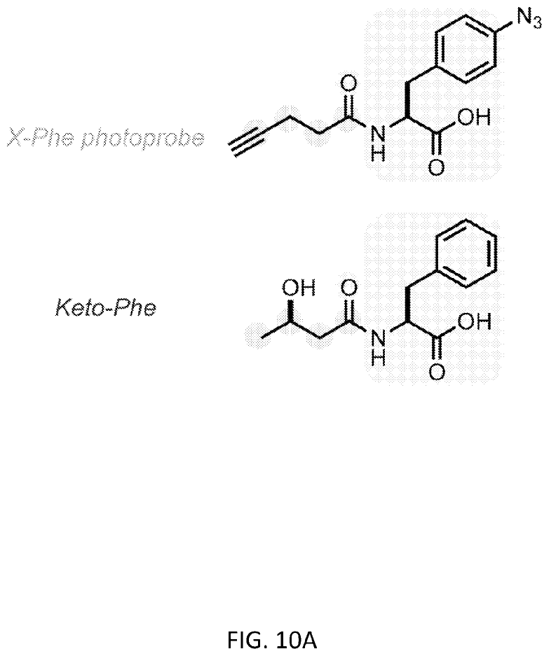

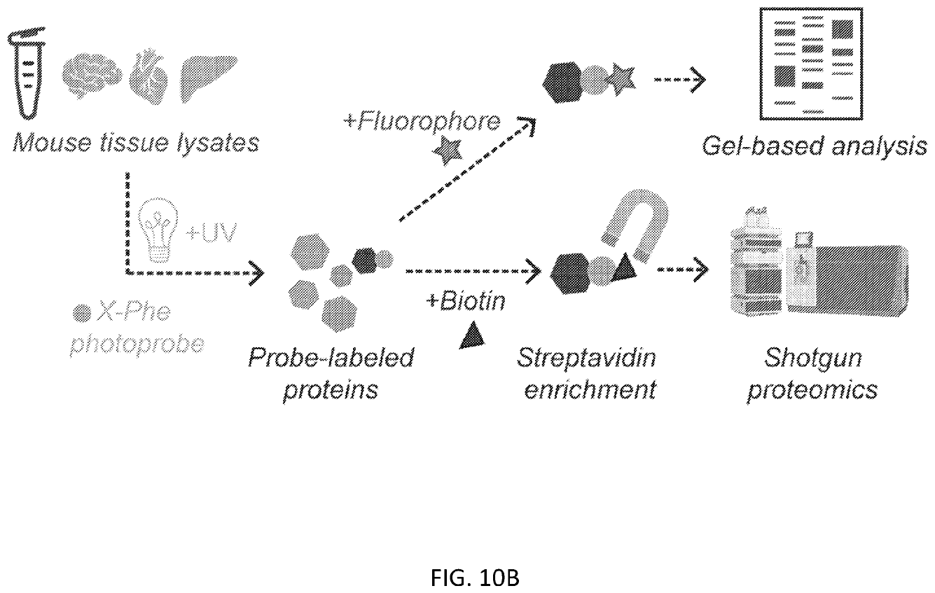

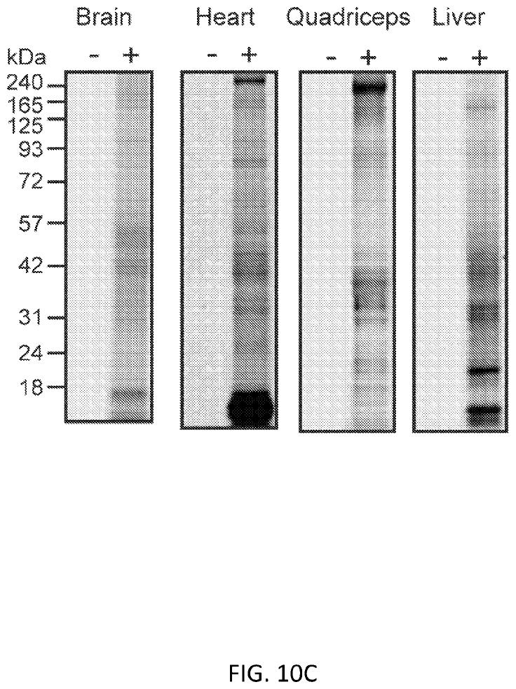

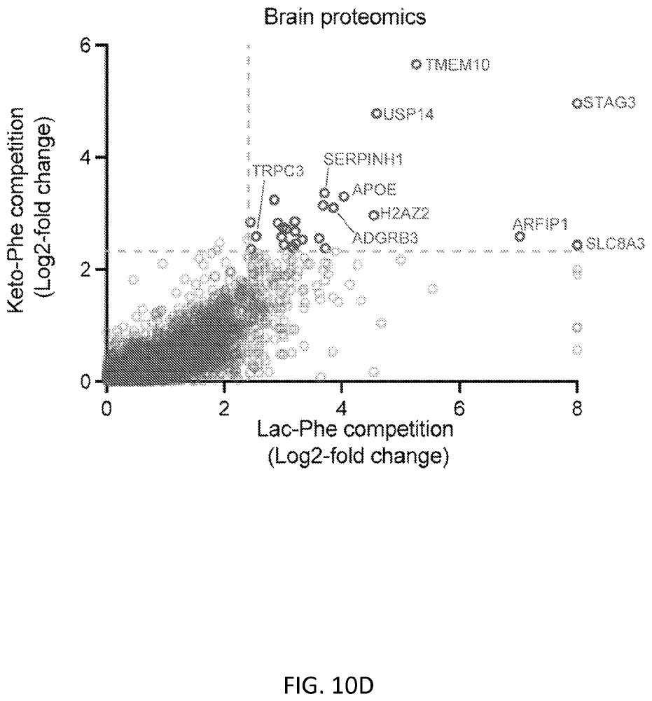

The functions of keto amino acids, if any, remain unknown. One possibility is that keto amino acids function as a metabolic reservoir for BHB storage. However, arguing against a metabolic intermediate hypothesis is the observation that genetic depletion of keto amino acids biosynthesis is not associated with a concomitant increase in total BHB levels (FIG. 9). mA second possibility is that keto amino acids might function as signaling metabolites. This hypothesis was based on the structural similarity of keto amino acids with other mammalian and bacterial signaling metabolites based on N-acylated amino acids, such as N-fatty acyl amino acids (Long, et al., Cell 2016 166:424-435), N-lactoyl amino acids (Li, et al., Nature 2022 606:785-790), and acylhomoserine lactones (Jemielita, et al., Elife 2018 166:424-435). To test this possibility, a chemoproteomic approach (Niphakis, et al., Cell 2015 161:1668-1680) was used to map direct protein targets of keto amino acids. “X-Phe protoprobe” was synthesized by direct amide coupling of azidophenylalanine with butynoic acid (FIG. 10A). The X-Phe photoprobe was designed to share many structural aspects in common with Keto-Phe, including the presence of a phenylalanine amino acid, an amide bond, and a short alkyl tail. Importantly, unlike the endogenous metabolites, X-Phe photoprobe also contained two synthetic functional groups, an azide and a terminal alkyne, which could enable downstream visualization, enrichment, and profiling of X-Phe labeled proteins directly in complex tissue lysates (FIG. 10B).

To determine if X-Phe photoprobe could directly label protein targets, whole tissue homogenized lysates were prepared from a panel of mouse metabolic tissues including brain, heart, quadriceps muscle, and liver. X-Phe photoprobe was directly incubated with each tissue lysate and the mixture was irradiated with UV light. After crosslinking, X-Phe photoprobe-labeled proteins were visualized by conjugation of a rhodamine azide fluorophore via click chemistry and then in gel-fluorescence detection. As shown in FIG. 10C, discrete bands corresponding to proteins crosslinked by X-Phe photoprobe were identified in each of the tissues. These protein bands were absent when the X-Phe photoprobe was not present in the UV crosslinking reaction (left lanes). Lastly, the protein banding pattern differed depending on the tissue lysate. In conclusion, the X-Phe photoprobe directly interacts with protein targets in a tissue-specific manner.

Because of the well-established roles of BHB in the brain, and the reported effects of ketones on cognition and behavior, total brain lysate was selected as a representative tissue source to identify X-Phe photoprobe-labeled proteins. Total brain lysate was separated by centrifugation into a supernatant and pellet fraction to enable broader downstream proteome coverage. UV-mediated crosslinking was performed in each of the two fractions, probe-labeled proteins were conjugated to biotin-azide via click chemistry, and then enriched on streptavidin beads. To identify interactions probe-protein that could be competed in the presence of excess “cold” competitor, probe pulldown experiments were performed in which 50-fold excess Keto-Phe was included in the UV-mediated crosslinking step. Because Lac-Phe and Keto-Phe are structural congeners, as an additional comparison, pulldown experiments were performed in which 50-fold Lac-Phe was included as a competitor. Peptides recovered after on-bead trypsin digestion were labeled with tandem mass tags and analyzed by quantitative shotgun proteomics.

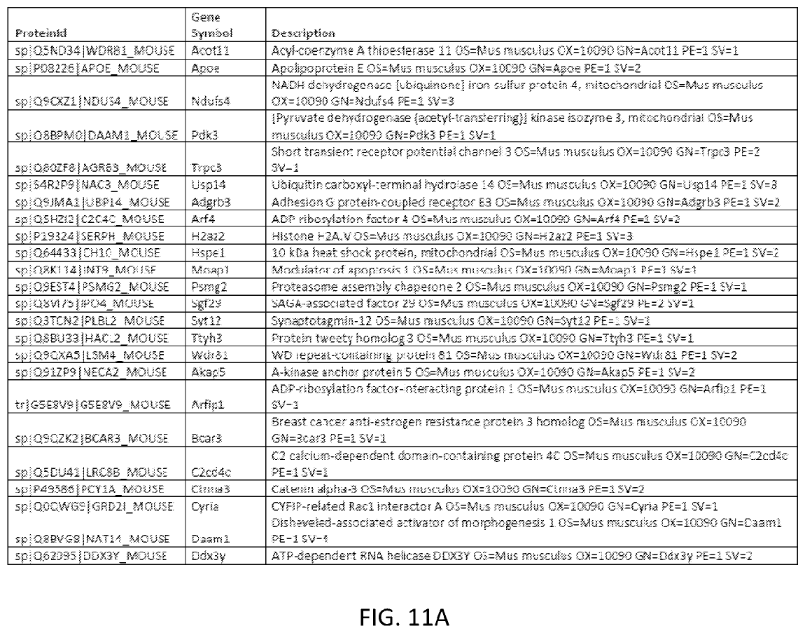

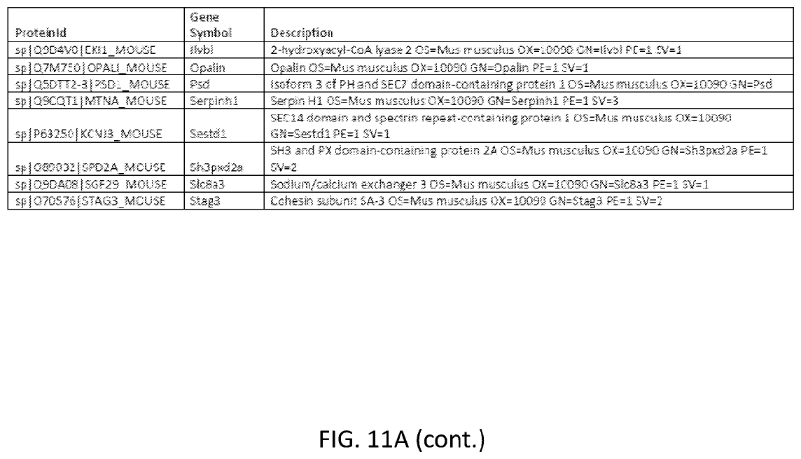

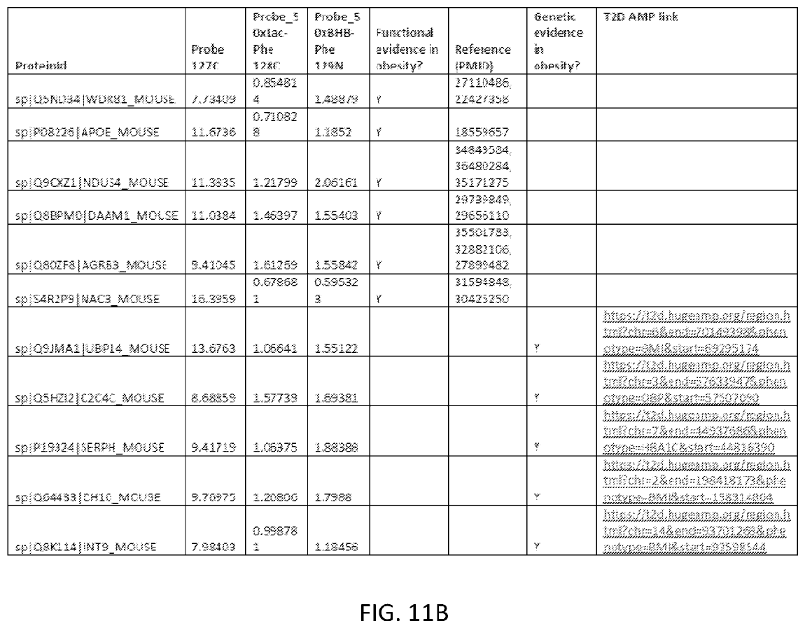

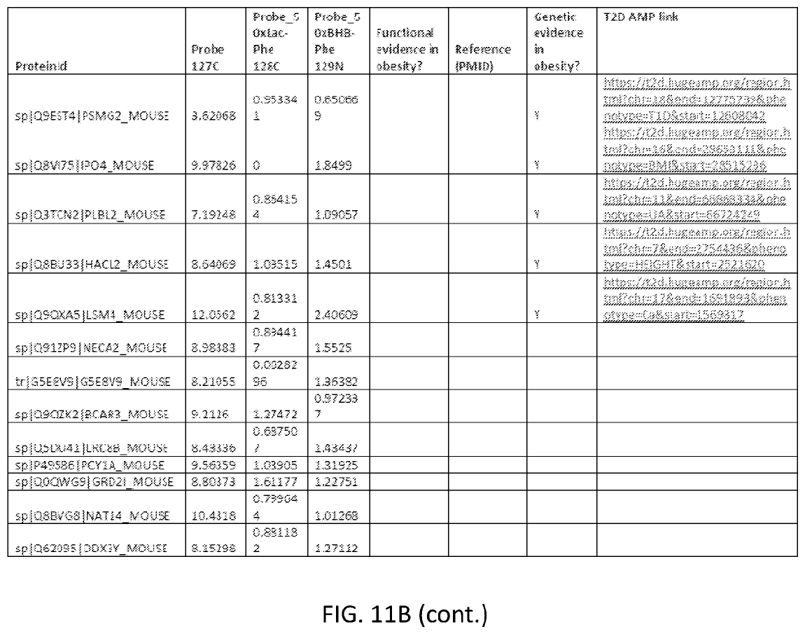

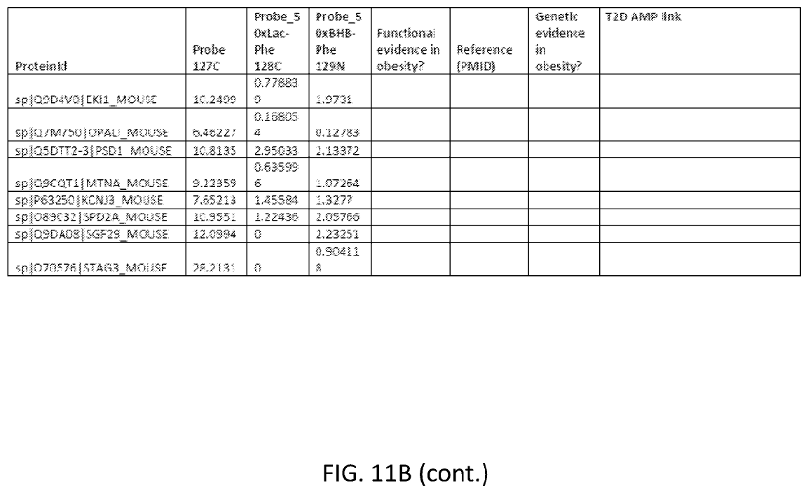

A total of 32 brain proteins that were both crosslinked by X-Phe photoprobe and also >80% competed by “cold” Keto-Phe were identified (FIG. 10D and FIGS. 11A-11B). Remarkably, the vast majority of these protein targets (30 out of 32, 94%) were also >80% competed by Lac-Phe (FIGS. 11A-11B), demonstrating that lactoyl and keto amino acids can directly bind to an overlapping set of protein targets in the brain. Brain keto amino acid binding proteins were localized to diverse and nearly all subcellular compartments, and included cell surface receptors (e.g., TRPC3, ADGRB3); disease-associated proteins (e.g., APOE); neurotransmission-related proteins (e.g., SYT12); and mitochondrial proteins (e.g., NDUFS4). This data demonstrates that keto amino acids can directly bind to multiple proteins in tissue lysates in vitro.

### Example 7—Keto Amino Acids Suppress Food Intake and Body Weight

Of the 32 BHB-amino acid protein targets in the brain, 6 proteins had previous experimental data demonstrating a functional role in obesity and/or body weight regulation in mouse studies (FIGS. 11A-11B). An additional 10 proteins were encoded by genes associated with body mass index or adiposity traits in human genome-wide association studies (FIGS. 11A-11B). This data raised the intriguing possibility that keto amino acids may also be bioactive ketone metabolites that also regulate energy homeostasis, potentially via direct binding and modulating of one or multiple of these protein targets. Previous studies showed that oral supplementation of high doses of BHB acutely suppresses appetite in humans (Stubbs, et al., Obesity 2018 26:269-273) and reduces body weight in obese mice (Deemer, et al., Obesity 2020 28:1447-1455). However, whether these effects are due to BHB alone, or some combination of BHB and downstream BHB-derived molecules such as the keto amino acids, has not been explored. Further supporting this idea, nearly all of the X-Phe photoprobe labeled, proteins competed by Keto-Phe were also competed by Lac-Phe, demonstrating convergence of the binding targets for Keto-Phe and anorexigenic Lac-Phe (FIG. 10D and FIGS. 11A-11B).

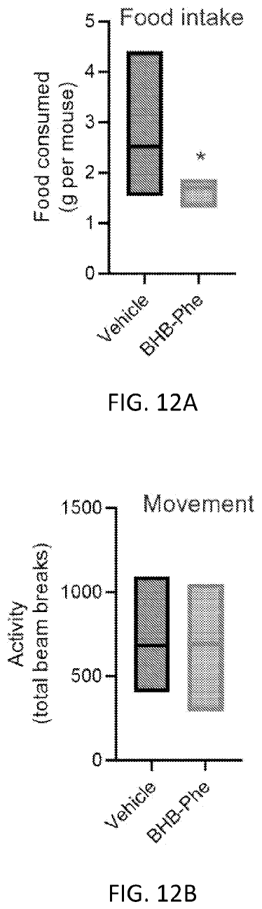

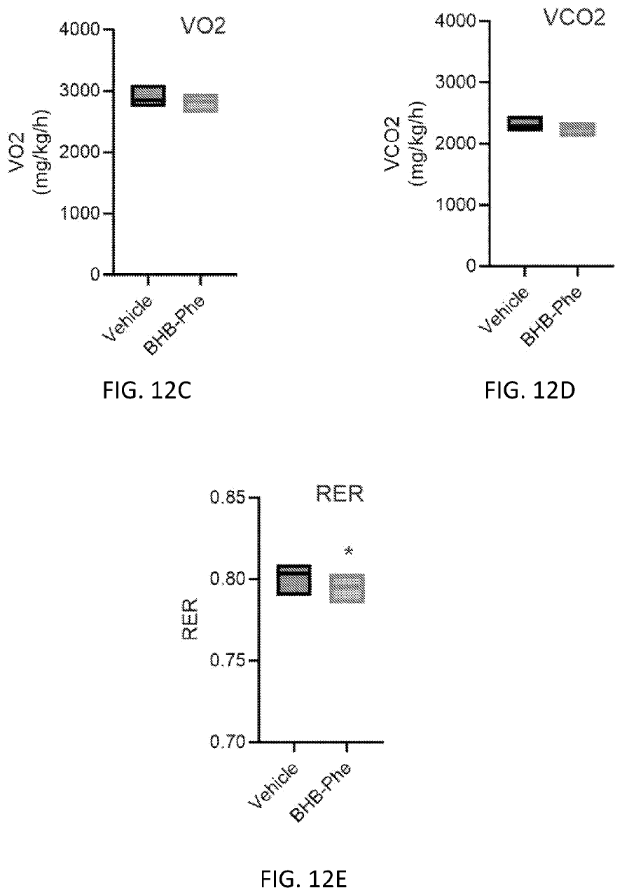

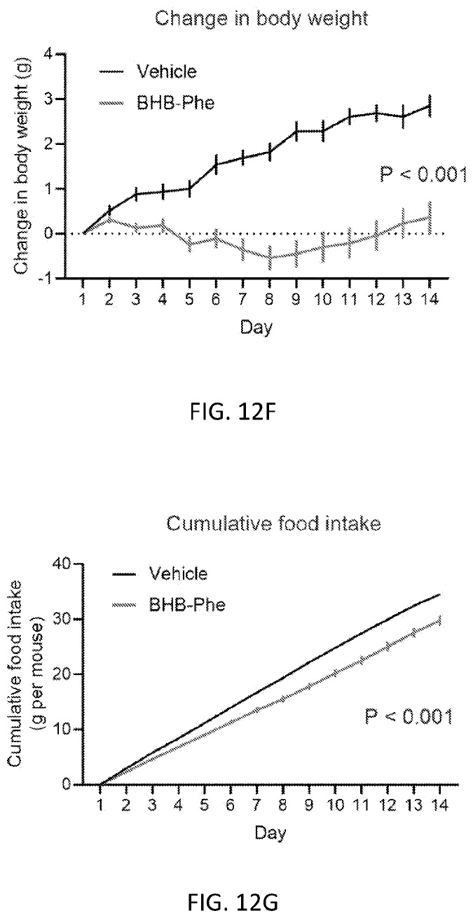

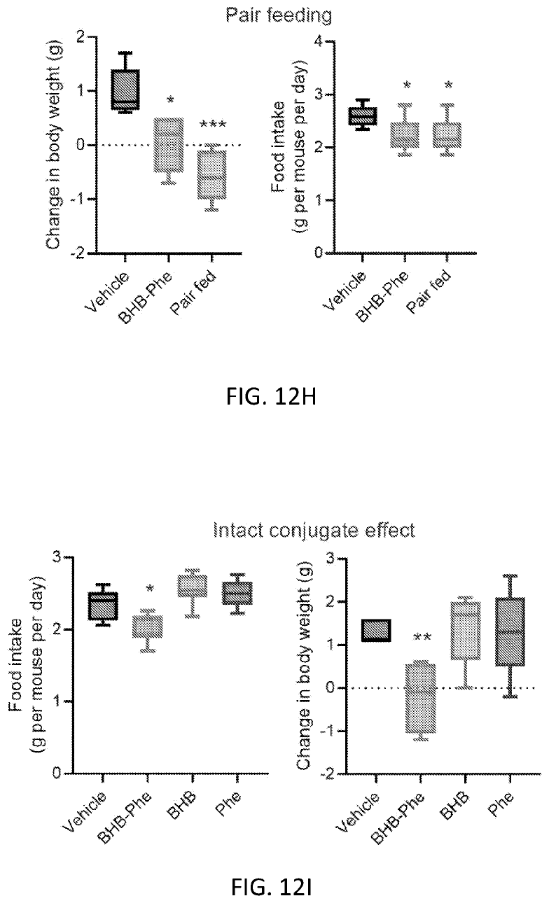

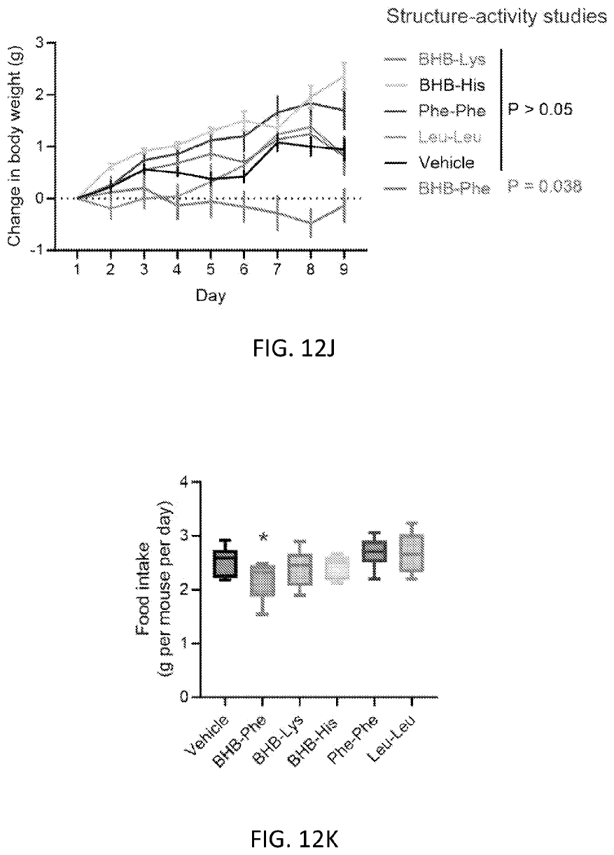

To directly test the potential bioactivity of keto amino acids in energy balance, the acute effects of Keto-Phe administration were determined in diet-induced obese (DIO) mice using metabolic chambers. Keto-Phe was initially selected because this metabolite exhibited the most chemical similarity with exercise-inducible Lac-Phe. A single administration of Keto-Phe (50 mg/kg, intraperitoneal [IP]) to mice reduced food intake by ~30% over a 10 h period (FIG. 12A). Importantly, over this time period no effects of Keto-Phe on ambulatory movement (FIG. 12B), energy expenditure (VO2 consumption and VCO₂ production), or respiratory exchange ratio were observed (RER) (FIGS. 12C-12E). Therefore, Keto-Phe exhibits acute hypophagic activity without modulating pathways of energy expenditure.

To determine if the suppressive effects of Keto-Phe on food intake would be sufficiently sustained to produce reduced body weight, chronic injections of Keto-Phe (50 mg/kg/day, IP) to DIO mice were performed over a 10-day period. Body weight and food intake were monitored daily. Over this period, Keto-Phe treatment resulted in a durable suppression of daily food intake by an average of −30% (FIG. 12G). Consequently, and as expected, daily Keto-Phe treatment also resulted in a concomitant reduction in body weight gain over the experimental period (FIG. 12F), demonstrating that long-term administration of keto amino acids is sufficient to reduce obesity and body weight.

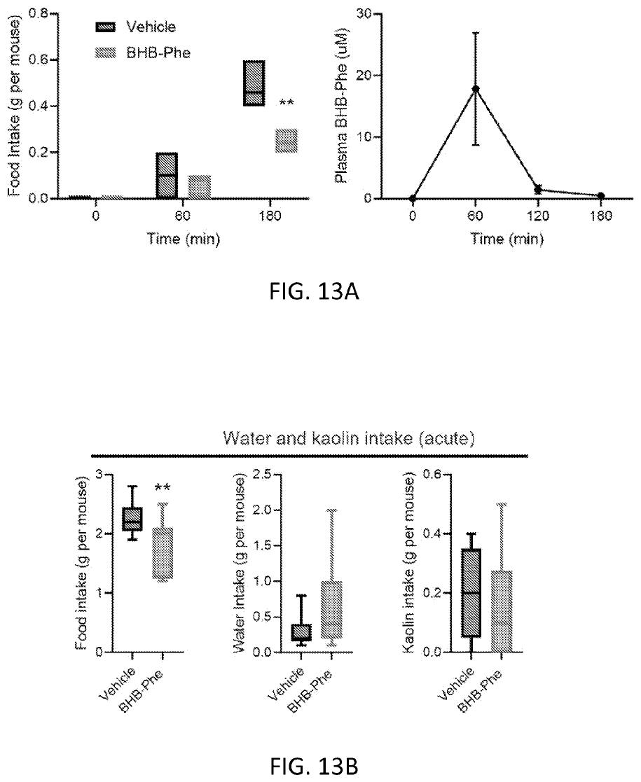

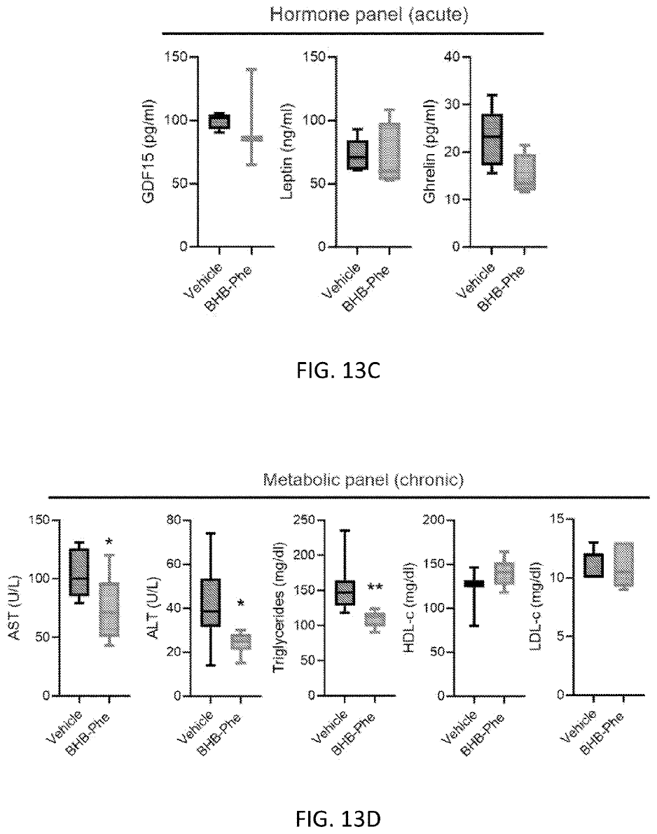

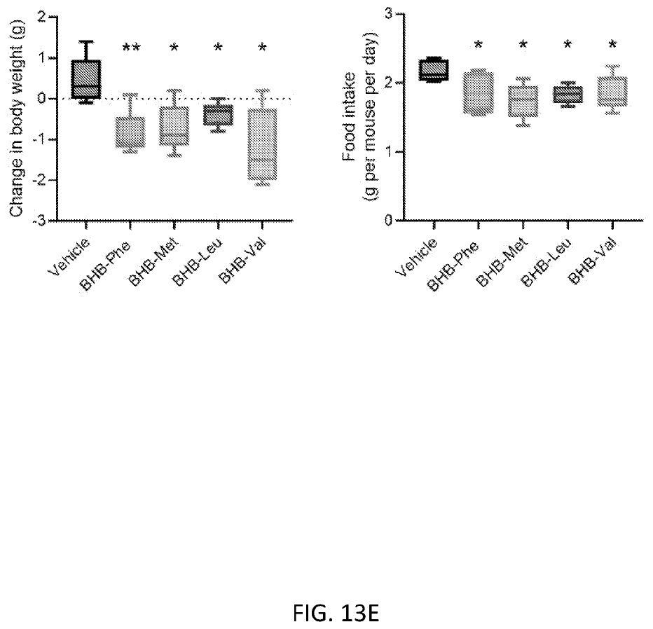

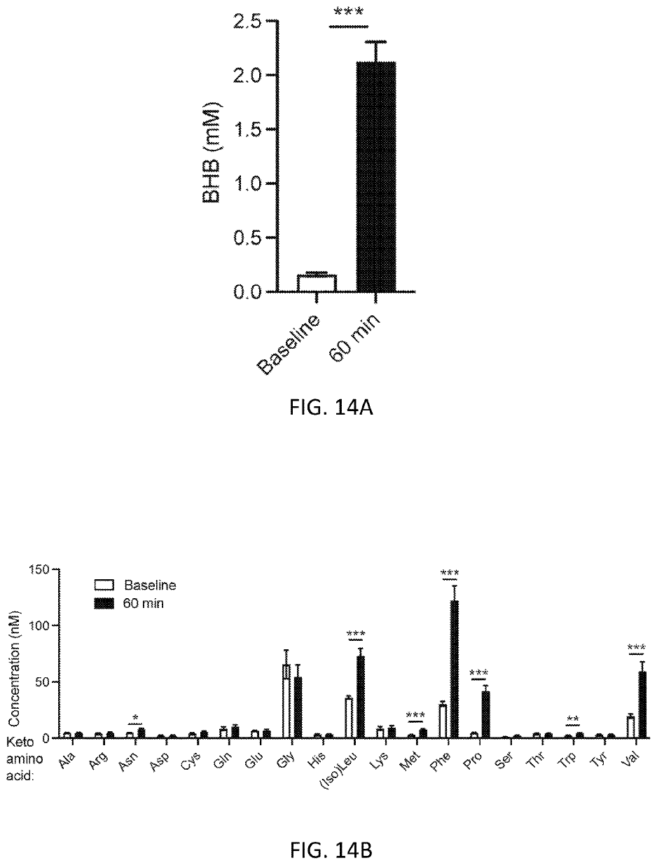

Lastly, to determine whether the hypophagic and anti-obesity effects of Keto-Phe are unique to this specific metabolite or shared across the class of keto amino acids, large quantities of several other keto amino acids, including Keto-Met, Keto-Leu, and Keto-Val were synthesized. These molecules were tested in a chronic, head-to-head experiment with Keto-Phe. Administration of any of the keto amino acids was effective in suppressing body weight compared with vehicle-treated mice, with approximately similar magnitude of effects (FIG. 13E). In conclusion, keto amino acids are bioactive metabolites that suppress food intake and body weight when administered to mice.

While studies shown herein only examined the role of keto amino acids in the context of energy homeostasis, the physiologic functions of keto amino acids may extend to other physiologic pathways as well. For instance, ketosis is being explored in a variety of other contexts, such as in neurodegenerative diseases (Fortier, at al., Alzheimer's Dement. 2021 17:543-552), inflammation (Youm, et al., Nat. Med. 2015 21:263-269), muscle resilience (Benjamin, et al., Cell Metab. 2022 34:902-918.e6), cancer treatment (Dmitrieva-Posocco, et al., Nature 2022 605:160-165), and several other age-associated diseases. In these disease paradigms, BHB itself has been proposed to be a key molecular effector of ketosis. The data shown herein demonstrates that keto amino acids are also produced when levels of BHB are high, raising the possibility that the effects of ketosis and BHB in these other contexts might also be, at least in part, mediated by concomitant production of keto amino acids.

BHB-Phe is a congener of Lac-Phe and both share chemical similarity as well as a common biosynthetic pathway via CNDP2. Therefore, it was possible that BHB-Phe might function as a ketosis-inducible metabolite that regulates body weight. First, gain-of-function approaches were used to determine if BHB-Phe is sufficient to reduce food intake and body weight. In an initial study of DIO mice in home cages, it was found that a single administration of synthetic BHB-Phe (50 mg/kg, IP) suppressed food intake at a 3 h, but not 1 h time point (FIG. 13A). Plasma BHB-Phe levels in this same experiment peaked at 1 h, and returned to largely baseline values by 3 h (FIG. 13A). In a second cohort of DIO mice in metabolic chambers, BHB-Phe (50 mg/kg, IP) reduced food intake without affecting movement, oxygen consumption or CO² production (FIGS. 12A-12D). A reduction in respiratory exchange ratio (RER) was also observed, consistent with a suppression of food intake (FIG. 12E). In a third experiment, it was found that acute administration of BHB-Phe (50 mg/kg, IP) suppressed food intake without affecting water intake or kaolin consumption, establishing specific suppression of food ingestion (FIG. 13B). Lastly, plasma levels of other feeding-regulating hormones, such as ghrelin, leptin, and GDF15, were also unaltered in mice following a single administration with BHB-Phe (50 mg/kg, IP, FIG. 13C).

In chronic studies over a 2-week period, BHB-Phe treatment (50 mg/kg/day, IP) resulted in a durable suppression of daily food intake and, as expected, a concomitant reduction in body weight gain (FIGS. 12F-12G). At the end of the experiment, BHB-Phe-treated mice exhibited reductions in AST, ALT, and total triglycerides (TG); no changes were found in HDL-or LDL-cholesterol (FIG. 13G). BHB-Phe-treated mice lost the same amount of weight as pair-fed controls (FIG. 12H), demonstrating that the observed suppression of food intake explains the observed change in body weight in BHB-Phe-treated mice.

Many additional in vivo experiments were performed to understand the structural requirements of BHB-Phe that were important for its body weight-lowering effects. First, only BHB-Phe (50 mg/kg/day, IP) efficiently suppressed body weight and food intake, whereas BHB alone or phenylalanine alone at the same doses were without effect (FIG. 121), demonstrating a requirement for the intact conjugate. Similarly, only BHB-Phe suppressed food intake and body weight, whereas the related metabolites BHB-Lys and BHB-His (50 mg/kg, IP), as well as the dipeptides Phe-Phe (50 mg/kg, IP) and Leu-Leu (50 mg/kg, IP), failed to reduce food intake or body weight (FIGS. 12J-12K). Lastly, BHB-Phe, BHB-Val, BHB-Leu, and BHB-Met (50 mg/kg, IP, each) exhibited similar anorexigenic and body weight-lowering effects in vivo (FIG. 13E). Therefore, administration of BHB-conjugated to specific hydrophobic amino acids is sufficient to reduce food intake and body weight in vivo, while other BHB-conjugated metabolites, and other dipeptides, do not have any effects in these same assays.

### Example 8—Conservation and Ketosis-Inducibility of Keto Amino Acids in Humans

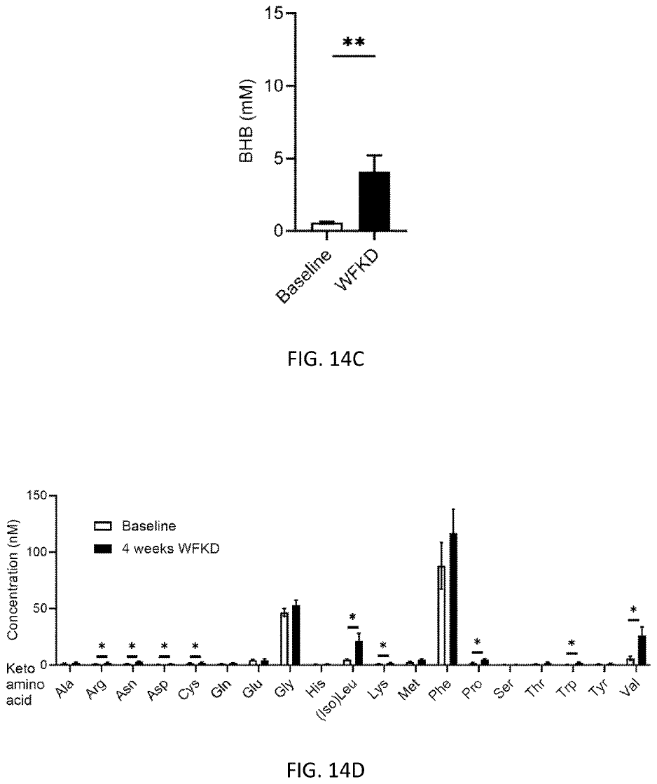

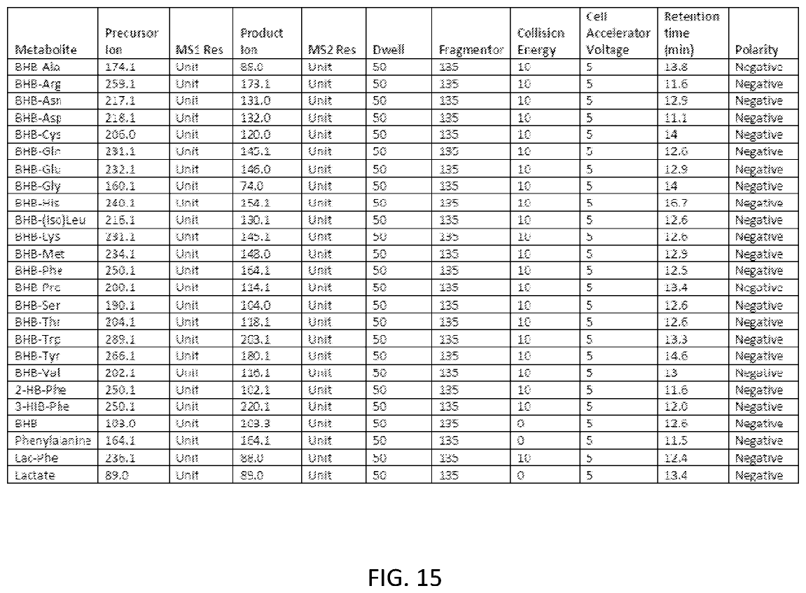

To determine if keto amino acids are also endogenous and ketosis-inducible metabolites in humans, keto amino acids were measured in plasma from two independent human cohorts subjected to two different ketosis stimuli. The first cohort consisted of participants from a trial of exogenous ketone supplementation (ClinicalTrials.gov ID NCT04194450). The cohort examined here (N=7) constituted a random sub-sample of participants from the overall study. After fasting for 24 h, participants consumed a ketone monoester drink (0.3 g/kg HVMN Ketone Ester). Plasma was collected at baseline and also at 60 min post ketone drink. As expected, BHB levels were dramatically elevated following ketone ester drink consumption (FIG. 14A). Many keto amino acids were also detectable in baseline plasma samples and dramatically elevated after ketone ester drink, including elevation of Keto-(iso) Leu (2-fold), Keto-Phe (3-fold), and Keto-Val (2.5-fold) (FIG. 14B).

In a second human cohort, plasma previously isolated from individuals in the Keto-Med interventional trial was used (N=12). This study was a single site, randomized clinical trial where individuals with prediabetes or type 2 diabetes followed the well-formulated ketogenic diet (WFKD). A random sub-sample of participants from this study were examined. Individuals on WFKD were counseled to sustain nutritional ketosis by limiting carbohydrates to 20-50 g/day and keeping proteins to 1.5 g/kg ideal body weight per day, with the remaining calories from fats. 4 weeks of dietary intervention was selected as the time point for plasma metabolomics analysis because food was directly delivered to the participants for the first four weeks and therefore the diet was better controlled. Individuals on the WFKD exhibited 5-fold elevated BHB levels compared to their own baseline levels (FIG. 14C). Robust induction of a wide spectrum of keto amino acids was observed following four weeks of WFKD, some of which overlapped with the ketone ester drink cohort, such as Keto-(iso) Leu and Keto-Val, and several others that had not been observed to be induced by ketone ester drink (e.g., Keto-Gln and Keto-Thr, FIG. 14D). In conclusion, keto amino acids are endogenously present in humans and induced following pharmacological or nutritional ketosis.

### Materials and Methods

Cell line cultures. All cell lines were obtained from ATCC and grown at 37° C. with 5% CO². HEK293T and RAW264 cells were grown in Dulbecco's modified Eagle's medium (DMEM) with 10% fetal bovine serum (FBS) and penicillin/streptomycin (pen/strep). All cell lines were used directly from ATCC vials and were not specifically authenticated for this study. Cell lines were negative upon testing for mycoplasma contamination

General animal information. Animal experiments were performed according to procedure approved by the Stanford University Administrative Panel on Laboratory Animal Care (APLAC). Mice were maintained in 12-hr light-dark cycles at 22° C. and ~50% relative humidity and fed a standard irradiated rodent chow diet. Where indicated, high-fat diet (D12492, Research Diets 60% kcal from fat) and ketogenic diet (D06040601, Research Diets, 80% kcal fat/15% kcal protein/5% kcal carbohydrate) was used. Male C57BL/6J (stock no. 000664) and male C57BL/6J DIO mice (stock no. 380050) were purchased from Jackson Laboratory. Male C57BL/6NCrl (stock no. 027) mice were purchased from Charles River Laboratory. Whole body CNDP2-KO mice (catalog number, C57BL/6NCrl-Cndp2em1 (IMPC) Mbp/Mmucd, RRID: MMRRC_043492-UCD) were obtained from the Mutant Mouse Regional Resource Center, a NCRR-NIH funded strain repository. For in vivo injection of mice with metabolites, compounds were dissolved in 18:1:1 (by volume) of saline/Kolliphor EL (Sigma Aldrich)/DMSO. Compounds were administered to mice daily via intraperitoneal injections at 5 μl/g body weight at the indicated doses. For all injection experiments, mice were mock injected with the vehicle for 3-5 days until body weights have stabilized. Sample sizes were determined on the basis of previous experiments using similar methodologies. For metabolite administration experiments, mice were randomly assigned to treatment groups. Experimenters were not blinded to groups.

Chemicals. The full inventory of commercially available compounds, purchased from Fisher, Sigma, Alfa Aesar, TCI, Mallinckrodt, United States Biochemical Corporation, Thermo and Acros, is described in Key Resources Table. The synthesis of Keto-Phe, Keto-Val, Keto-Leu and Keto-Met are described below.

CNDP2 active site modeling with BHB and lactate. Molecular docking and visualization was performed using Autodock Vina (Eberhardt, et al., J. Chem. Inf. Model. 2021 61:3891-3898) through UCSF Chimera software (Pettersen, et al., J. Comput. Chem. 2004 25:1605-1612) (rbvi.ucsf.edu/chimera). The human CNDP2 structure in complex with bestatin was downloaded from PDB (PDB #4RUH, 4). The bestatin molecule was removed prior to docking simulation, however the two Mn2+ ions that serve as cofactors in the binding pocket were not removed. The whole protein was given as the search area for the model, the default options were used as parameters, the number of binding modes given was 5, exhaustiveness of search was 8, and maximum energy difference was 3. The images shown represent the docking site with highest modeled affinity for the ligand.

Synthesis of Keto-Phe. 1-ethyl-3-(3-dimethylaminopropyl) carbodiimide (1.05 eq.) was added to a cold mixture of sodium (R)-3-hydroxybutyrate (1 eq.) and 1-hydroxybenzotriazole (1 eq.) in dichloromethane (0.2 M) under argon. After 10 min, L-phenylalanine ethyl ester hydrochloride (1 eq.) and N,N-diisopropylethylamine (3 eq.) in dichloromethane (0.2 M) were added to the mixture. The ice bath was removed and the reaction was stirred for 16 h under argon at ambient temperature. One-third of the solvent was then removed and the dichloromethane solution was washed with 5% HCl, 5% NaHCO₃ and saturated NaCl solutions. The organic layer was dried over MgSO₄, filtered and concentrated. The resulting crude product was purified by column chromatography, eluting with ethyl acetate/hexane to afford the N-betahydroxybutyryl phenylalanine ethyl ester. The above ester was dissolved in tetrahydrofuran (0.5 M) and 2 N LiOH (2 eq.) was added. The resulting mixture was stirred at room temperature for 2 h. The solvent was then removed and the resulting residue was dissolved in water and acidified with 5% HCl to pH 3.

The resulting mixture was extracted with ethyl acetate three times, and the combined organic layers were washed with saturated NaCl solution. The organic layer was dried over MgSO₄, filtered, concentrated, coevaporated three times with DCM and then dried under high vacuum to give BHB-Phe (N-betahydroxybutyryl-phenylalanine) as a white powder. 1H NMR (400 MHZ, Methanol-d4): 0 7.23 (dq, J=16.1, 8.6, 7.9 Hz, 5H), 4.66 (dd, J=8.5, 5.2 Hz, 1H), 4.01 (dt, J=12.5, 6.2 Hz, 1H), 3.19 (dd, J=13.9, 5.1 Hz, 1H), 2.95 (dd, J=13.9, 8.8 Hz, 1H), 2.28 (qd, J=14.2, 6.5 Hz, 2H), 1.16-1.06 (m, 3H). MS (m/z): 250.108 [M−H].

Synthesis of Keto-Val. 1-ethyl-3-(3-dimethylaminopropyl) carbodiimide (1.05 eq.) was added to a cold mixture of sodium (R)-3-hydroxybutyrate (1 eq.) and 1-hydroxybenzotriazole (1 eq.) in dichloromethane (0.2 M) under argon. After 10 min, L-valine ethyl ester hydrochloride (1 eq.) and N,N-diisopropylethylamine (3 eq.) in dichloromethane (0.2M) were added to the mixture. The ice bath was removed and the reaction was stirred for 16 h under argon at ambient temperature. One-third of the solvent was then removed and the dichloromethane solution was washed with 5% HCl, 5% NaHCO₃ and saturated NaCl solutions. The organic layer was dried over MgSO₄, filtered and concentrated. The resulting crude product was purified by column chromatography, eluting with ethyl acetate/hexane to afford the N-betahydroxybutyryl valine ethyl ester. The above ester was dissolved in tetrahydrofuran (0.5 M) and 2 N LiOH (2 eq.) was added. The resulting mixture was stirred at room temperature for 2 h. The solvent was then removed and the resulting residue was dissolved in water and acidified with 5% HCl to pH 3. The resulting mixture was extracted with ethyl acetate three times, and the combined organic layers were washed with saturated NaCl solution. The organic layer was dried over MgSO₄, filtered, concentrated, coevaporated three times with DCM and then dried under high vacuum to give Keto-Val (N-betahydroxybutyryl-valine) as a colorless syrup. 1H NMR (400 MHZ, Methanol-d4): Õ 4.29 (d, J=5.3 Hz, 1H), 4.09 (dq, J=12.4, 6.1 Hz, 1H), 2.35 (qt, J=14.2, 6.5 Hz, 2H), 2.12 (dq, J=13.5, 6.8 Hz, 1H), 1.17 (d, J=6.2 Hz, 3H), 1.00-0.86 (m, 6H). MS (m/z): 202.108 [M−H].

Synthesis of Keto-Leu. 1-ethyl-3-(3-dimethylaminopropyl) carbodiimide (1.05 eq.) was added to a cold mixture of sodium (R)-3-hydroxybutyrate (1 eq.) and 1-hydroxybenzotriazole (1 eq.) in dichloromethane (0.2 M) under argon. After 10 min, L-leucine ethyl ester hydrochloride (1 eq.) and N,N-diisopropylethylamine (3 eq.) in dichloromethane (0.2M) were added to the mixture. The ice bath was removed and the reaction was stirred for 16 h under argon at ambient temperature. One-third of the solvent was then removed and the dichloromethane solution was washed with 5% HCl, 5% NaHCO₃ and saturated NaCl solutions. The organic layer was dried over MgSO4, filtered and concentrated. The resulting crude product was purified by column chromatography, eluting with ethyl acetate/hexane to afford the N-betahydroxybutyryl leucine ethyl ester. The above ester was dissolved in tetrahydrofuran (0.5 M) and 2 N LiOH (2 eq.) was added. The resulting mixture was stirred at room temperature for 2 h. The solvent was then removed and the resulting residue was dissolved in water and acidified with 5% HCl to pH 3. The resulting mixture was extracted with ethyl acetate three times, and the combined organic layers were washed with saturated NaCl solution. The organic layer was dried over MgSO₄, filtered, concentrated, coevaporated three times with DCM and then dried under high vacuum to give Keto-Leu (N-betahydroxybutyryl-leucine) as a colorless syrup. 1H NMR (400 MHZ, Methanol-d4): 1H NMR (400 MHZ, Methanol-d4) δ 4.47-4.39 (m, 1H), 4.19-4.08 (m, 1H), 2.43-2.29 (m, 2H), 1.73 (dp, J=13.0, 6.5 Hz, 1H), 1.67-1.58 (m, 2H), 1.21 (d, J=6.2 Hz, 3H), 0.95 (dd, J=13.6, 6.5 Hz, 6H). MS (m/z): 216.124 [M−H]⁻.

Synthesis of Keto-Met. 1-ethyl-3-(3-dimethylaminopropyl) carbodiimide (1.05 eq.) was added to a cold mixture of sodium (R)-3-hydroxybutyrate (1 eq.) and 1-hydroxybenzotriazole (1 eq.) in dichloromethane (0.2 M) under argon. After 10 min, L-methionine ethyl ester hydrochloride (1 eq.) and N,N-diisopropylethylamine (3 eq.) in dichloromethane (0.2 M) were added to the mixture. The ice bath was removed and the reaction was stirred for 16 h under argon at ambient temperature. One-third of the solvent was then removed and the dichloromethane solution was washed with 5% HCl, 5% NaHCO₃ and saturated NaCl solutions. The organic layer was dried over MgSO₄, filtered and concentrated. The resulting crude product was purified by column chromatography, eluting with ethyl acetate/hexane to afford the N-betahydroxybutyryl methionine ethyl ester. The above ester was dissolved in tetrahydrofuran (0.5 M) and 2 N LiOH (2 eq.) was added. The resulting mixture was stirred at room temperature for 2 h. The solvent was then removed and the resulting residue was dissolved in water and acidified with 5% HCl to pH 3. The resulting mixture was extracted with ethyl acetate three times, and the combined organic layers were washed with saturated NaCl solution. The organic layer was dried over MgSO₄, filtered, concentrated, coevaporated three times with DCM and then dried under high vacuum to give Keto-Met (N-betahydroxybutyryl-methionine) as a white powder. 1H NMR (400 MHZ, Methanol-d4): δ 4.62-4.45 (m, 1H), 4.12 (d, J=8.6 Hz, 1H), 2.66-2.44 (m, 2H), 2.45-2.24 (m, 2H), 2.23 -1.81 (m, 5H), 1.28-1.08 (m, 3H). MS (m/z): 234.080 [M−H]⁻.

Synthesis of X-Phe photoprobe. To a stirred solution of 4-pentynoic acid (50 mg, 0.51 mmol) in ethyl acetate (5 mL) was added EDC.HCl (97 mg, 0.51 mmol) at 0 °C. The reaction mixture was stirred for 10 mins and perfluorophenol (93 mg, 0.51 mmol) was added to the reaction mixture. The resulting mixture was stirred at RT for 14 h. The mixture was concentrated. The crude product was purified by Biotage to give an intermediate, perfluorophenyl pent-4-ynoate (68 mg, 50%). LC-MS (ESI) m/z 264.75 [M+H]+. To a stirred solution of phenyl azide (40 mg, 0.196 mmol) in THF (2 mL) and water (0.5 ml) at 0° C. was added perfluorophenyl pent-4-ynoate (62 mg, 0.235 mmol) and triethylamine (59 mg, 0.588 mmol). The resulting mixture was stirred at rt for 2 h. THF was removed in vacuo. The aqueous layer was washed with ether (2×5 mL), and acidified to pH 2 with 1N HCl, extracted with 10% methanol in DCM (3×20 mL). The organic layer was washed with water, brine and dried with sodium sulfate, filtered and concentrated in vacuo and dried to give X-Phe photoprobe (15 mg, 27%). ¹H NMR (400 MHZ, CDCl₃) 0 7.16 (d, J=8 Hz, 2H), 6.95 (d, J=8 Hz, 2H), 6.09 (d, 1H), 4.89-4.84 (q, 1H), 3.24-3.19 (m, 1H), 3.13-3.08 (m, 1H), 2.50-2.47 (m, 2H), 2.42-2.38, 1.95 (s, 1H). LC-MS (ESI) m/z 287.05 [M+H]+.

CNDP2 active site modeling with BHB and lactate. Molecular docking and visualization was performed using Autodock Vina31 through UCSF Chimera software32 (rbvi.ucsf.edu/chimera). The human CNDP2 structure in complex with bestatin was downloaded from PDB (PDB #4RUH, 4). The bestatin molecule was removed prior to docking simulation, however the two Mn2+ ions that serve as cofactors in the binding pocket were not removed. The whole protein was given as the search area for the model, the default options were used as parameters, the number of binding modes given was 5, exhaustiveness of search was 8, and maximum energy difference was 3. The images shown represent the docking site with highest modeled affinity for the ligand.

Human induced pluripotent stem cells (iPSCs) culture and cardiac differentiation into cardiomyocytes (CMs). Human iPSCs from healthy donors were provided by Stanford Cardiovascular Institute Biobank and cultured in the E8 medium. When iPSCs were grown to 95% confluence, the E8 medium was replaced with RPMI 1640 (GIBCO) supplemented with B27 minus insulin (GIBCO) and 5 UM CHIR-99021 (Selleck Chemicals) to start cardiac differentiation at day 0. On day 2, the medium was replaced with RPMI-B27 minus insulin. On day 3, the medium was changed to RPMI-B27 minus insulin containing 5 μM IWR-1 (Selleck Chemicals) and was maintained for 48 hours. On day 5, the medium was changed back to RPMI-B27 minus insulin and was maintained for 48 hours. Then, the cells were incubated with complete CM medium consisting of RPMI 1640 medium and B27 supplement plus insulin (GIBCO). On day 12, the medium was replaced with RPMI-B27 without D-glucose (GIBCO) for metabolic purification of CMs for 4 days. Then, the medium was changed to the complete CM medium and replaced every 2 days. Human iPSC-CMs were dissociated by TrypLE Express (GIBCO) and re-plated on Matrigel-coated 12-well plate for further experiments. Cultures were maintained at 37° C. in a humidified incubator with 5% (v/v) CO².

In vitro enzyme activity assays. HEK293T cells were transfected with GFP, CNDP2 or CNDP2-E166A using PolyFect (Qiagen 301105) according to the manufacturer's instructions. The medium was changed one day post-transfection. After an additional 24 h, the cells were harvested in PBS, lysed by sonication and centrifuged (10 min at 15,000 rpm) to remove debris. Supernatant was collected and protein concentrations were adjusted. The in vitro reactions were conducted in Eppendorf tubes with 100 μL of 1 mg/mL protein and 20 mM substrates for keto amino acid synthase activity assays and. Reactions were incubated for 1 h at 37° C. and 20 μl of 1 M HCl were added to acidify the medium and to protonate keto amino acids. Reactions were vortexed and 400 μL of ethyl acetate were added to each reaction. Reactions were vortexed for 30 s to extract keto amino acids into the organic layer and centrifuged at 4° C. for 10 min at 15,000 r.p.m. A total of 300 μl from the top layer was transferred to a new Eppendorf tube and dried down under a stream of nitrogen. The residue was re-suspended in 150 μl of an 2:1:1 mixture of acetonitrile: methanol: water. The mixture was centrifuged at 4° C. for 10 min at 15,000 r.p.m. and the supernatant was transferred to a LC-MS vial.

Cellular enzyme activity assays. Cells were plated in 12-well plates at 70-80% confluence. The next day, cells were washed two times with PBS and incubated in 0.5 ml serum-free medium. After overnight incubation, 400 μl of medium was removed and 20 μl of 1 M HCl were added to acidify the medium and to protonate keto amino acids. Ethyl acetate (400 μl) was added into each sample and reactions were vortexed for 30 s to extract keto amino acids into the organic layer. Reactions were centrifuged at 4° C. for 10 min at 15,000 r.p.m. and total of 300 μl from the top layer was transferred to a new Eppendorf tube and dried down under a stream of nitrogen. The residue was re-suspended in 150 μl of an 2:1:1 mixture of acetonitrile: methanol: water. The mixture was centrifuged at 4° C. for 10 min at 15,000 r.p.m. and the supernatant was transferred to a LC-MS vial. Cells were kept on ice to collect the lysate. PBS (150 μl) was added into each well and the cells scraped into an Eppendorf tube. This step was repeated again to ensure all cells were collected. Cells were then centrifuged at 4° C. for 10 min at 2,000g and the supernatant removed to obtain the cell pellet. A volume of 150 μl of a 2:1:1 mixture of acetonitrile: methanol:water mixture was used to lyse the cells and precipitate large proteins. The mixture was centrifuged at 4° C. for 10 min at 15,000 r.p.m. and the supernatant was transferred to a LC-MS vial.

Western blot analysis. Cells were collected and lysed by sonication in RIPA buffer containing 1:100 HALT protease inhibitor. Cell lysates were centrifuged at 4° C. for 10 minutes at 13,000 rpm to remove residual cell debris. Protein concentrations of the supernatant were normalized using the Pierce BCA protein assay kit and combined with 4× NuPAGE LDS Sample Buffer with 10 mM DTT. Samples were then boiled for 10 minutes at 95 °C. Prepared samples were run on a NuPAGE 4-12% Bis-Tris gel then transferred to nitrocellulose membranes. Blots were blocked for 30 minutes at room temperature in Odyssey blocking buffer. Primary antibodies (mouse anti-FLAG and rabbit anti-Beta-Tubulin) were added to Odyssey blocking buffer at a ratio of 1:1000. Blots were incubated in the indicated primary antibodies overnight while shaking at 4 °C. The following day, blots were washed 3 times with PBS-T, 10 minutes each before staining with the secondary antibody for 1 hour at room temperature. The secondary antibody used was a goat anti-rabbit antibody diluted in blocking buffer to a ratio of 1:10,000. Following secondary antibody staining, the blot was washed 3 times with PBS-T before being imaged with the Odyssey CLx Imaging System.

Preparation of plasma samples for LC-MS analysis. Plasma was collected from mice via a submandibular bleed into lithium heparin tubes (BD, 365985) and immediately transferred onto ice. The blood was centrifuged at 4° C. at 5000 rpm for 5 min and the top layer of plasma was aliquoted and frozen at −80 °C. To extract polar metabolites from plasma for LC-MS analysis, 150 μl of a 2:1 mixture of acetonitrile/methanol was added to 50 μl of plasma. The mixture was centrifuged at 4° C. for 10 min at 15,000 rpm and the supernatant was transferred to a LC-MS vial.

Targeted metabolomics. Targeted measurements were performed using an Agilent 6470 triple quadrupole LC-MS instrument. MS analysis was performed using AJS in negative mode. The AJS source parameters were set as follows: the dry gas temperature was set at 250° C. with a gas flow of 12 l/min and the nebulizer pressure at 25 psi; the sheath gas temperature was set to 300° C. with the sheath gas flow set at 12 l/min; and the capillary voltage was set to 3,500 V. Separation of polar metabolites was performed as described above in the ‘Untargeted measurements of metabolites by LC-MS’ section. Multiple reaction monitoring was performed for the indicated metabolites with the listed dwell times, fragmentor voltage, collision energies, cell accelerator voltages and polarities. The MS ionization parameters for the targeted metabolomics are presented in FIG. 15. Quantification of the endogenous metabolite concentrations were performed by generating a standard curve with known concentrations of each metabolite. Metabolite standards were analyzed alongside the plasma samples using the same targeted triple quadrupole LC/MS method. A calibration standard curve generated from the metabolite standard concentrations and total peak areas were used to calculate the concentrations of each endogenous metabolite. The dilution of the plasma during the sample preparation was also taken into account for the quantification.

Single cell mRNA analysis. Average mRNA expression for Oxct1, Hmgcl, and Cndp2 were directly based on the tissue-combined dataset with annotations (‘FACS_all. Robj”) 17 from figshare.com/articles/dataset/Robject_files_for_tissues_processed_by_Seurat/5821263/2?file=1 3092806 The heat map and hierarchical clustering of cell type-mRNA levels was performed with ClustVis (biit.cs.ut.ee/clustvis/).

Breeding and genotyping of CNDP2-KO mice. CNDP2-KO and wild type animals were obtained from the International Mouse Phenotyping Consortium (IMPC) and generated via heterozygous breeding crosses. Genotyping was performed as follows: Tail clippings were obtained from littermates and boiled for 30 minutes at 95° C. in 100 μl of 50 mM NaOH to extract genomic DNA. The solution was neutralized by adding 21 μl of 0.5 M Tris (pH 7.2). PCR reactions were performed by using primers for either the CNDP2 WT allele (Forward: 5′-CAGATGGCTCGGAGATACCAC-3′ (SEQ ID NO:1), Reverse: 5′-TTCCCGCTCCACCAAGGTGAAG-3′ (SEQ ID NO:2)) or CNDP2 KO allele (Forward: 5′-GCTCTGTAAGGGAAAGAGATGACCC-3′ (SEQ ID NO:3), Reverse: 5′-AATAGGACATACCCAGTTCTGTGAGG-3′ (SEQ ID NO:4)). The Promega GoTaq master mix was used for the PCR reaction. Each 25 μl reaction consisted of 12.5 μl of the promega master mix (M7122), 2.5 μl of a 10 UM mixture of forward and reverse primers, 2 μl of genomic DNA, and 8 μl of ultrapure water. The thermocycling program on BioRad C1000 Touch Thermo Cycler began with an initial 30 seconds at 95° C., followed by cycles of 30 seconds at 98° C., 30 seconds at 58° C., and 45 seconds at 72° C., followed by 5 minutes at 72° C. and finally held at 4 °C. PCR reactions for WT primers consisted of 30 cycles while PCR reactions for KO primers consisted of 48 cycles. Samples were run on a 2% agarose gel with 0.2 mg/ml EtBr. WT alleles are expected to yield a PCR product 160 base pairs in size while KO alleles are expected to yield PCR products that are 440 base pairs in size.

Alb-Hmgcl (−/−) mice. The HmgclAlb-Cre mice were generously provided by ELG and previously described.10 Mice were fed a ketogenic diet (89.5% of calories from fat, 10.4% of calories from protein, 0.1% of calories from carbohydrates; Research Diets D19042606) ad libitum for 48 hours, or fasted for 24 hours. Cre-negative littermates were used for controls.

In vitro crosslinking studies of X-Phe photoprobe. Tissues were collected from wild type C57BL/6J and homogenized using a bead blaster in PBS. The protein homogenate was filtered through a 40 μm strainer to remove any un-homogenized tissue and the lysate diluted to 2 mg/ml. Tissue lysate was incubated with 100 μM of the photoprobe and incubated on ice for 10 minutes protected from light. Reactions were then put under UV for 1 min on a UV Stratalinker. A TAMRA click reaction was performed by adding the following reagents to the indicated final concentrations: 100 mM TBTA, 1 mM CuSO4, 1 mM TCEP (freshly prepared), 25 μM TAMRA-N3. Reactions were allowed to proceed for 1 hour at room temp, protected from light. Loading buffer was added to the reactions, boiled for 10 minutes and run down a protein gel. The gel was imaged prior to staining with a coomassie dye.

Shotgun proteomics of X-Phe photoprobe-labeled proteins. Brains from wild type C57BL/6J mice were harvested and homogenized using a bead blaster in PBS. Brain homogenate was centrifuged (15,000 rpm, 10 min) to yield the cytosolic supernatant and membrane pellet fractions. The cytosolic fraction was transferred to a new eppie and centrifuged again (15,000 rpm, 10 min). This process was repeated 4 times until no pellet was present after centrifugation. The membrane pellet was washed with 1 ml PBS and centrifuged (15,000 rpm, 10 min); this step was also repeated 4 times to remove any cytosolic protein contamination. After the centrifugation steps, the membrane pellet was re-suspended in PBS and sonicated. For competition experiments, brain lysate was normalized to 2 mg/ml in PBS and incubated with competitors at the indicated concentrations at room temperature for 30 minutes. 100 μM of the photoprobe was added and the reactions incubated on ice for 10 minutes protected from light. Reactions were then put under UV for 1 min on a UV Stratalinker. A biotin click reaction was performed by adding the following reagents to the indicated final concentrations: 100 mM TBTA, 1 mM CuSO₄, 1 mM TCEP (freshly prepared), 30 μM Biotin-N3. Reactions were allowed to proceed for 1 hour at room temp, protected from light, and while rotating. The following prep was adapted from Niphakis et al. 2015. To each reaction, the following solvents were added in order, 4 ml cold methanol, 1 ml cold chloroform, 2 ml cold PBS. The mixture was vortexed well and centrifuged (4,600×g, 15 min, 4° C.). After centrifugation the organic and aqueous layers were removed without disruption of the protein disc. The protein disc was washed with a cold 1:1 mixture of methanol:chloroform 3 times. After the final wash, the protein disc was re-suspended in a 4:1 mixture of methanol:chloroform and sonicated. The insoluble protein was pelleted by centrifugation (4,600×g, 15 min, 4 C) and the pellet re-suspended in freshly prepared 500 μl of 6 M urea in PBS. 20 μl of 10% (w/v) SDS was added to the solution. The solution was then warmed in a 37 C water bath for 10 min to help dissolve poorly soluble protein. A streptavidin pulldown was the performed on the protein mixture. 250 μl of streptavidin beads were washed 3 times with 1 ml PBS. Protein samples were diluted with 5.5 ml of 0.25% SDS in PBS and incubated with streptavidin beads for 1.5 hr at room temperature. Following incubation, beads were washed 5 times each with 0.25% SDS in PBS, then PBS, then lastly with ultra-pure water Steptavidin beads were suspended in 150 μL resuspension buffer (1 M urea, 50 mM Tris-HCl, pH 8). Proteins were digested with 3 μg of trypsin for 4 hours. Digested peptides were reduced with 5 mM TCEP for 10 minutes. Cys residues were alkylated using 15 mM iodoacetimide (IAA) for 20 minutes. Finally, excess IAA was quenched with 10 mM DTT. Acidified digested peptides were desalted over C18 StageTips [PMID: 12585499]. Peptides were then eluted with 80% acetonitrile, 0.1% TFA and dried in a speedvac. Dried samples were reconstituted with 200 mM EPPS buffer, pH 8.0, and labelled with TMT reagents (Thermo Fisher Scientific). Following incubation at room temperature for 2 h, the reactions were quenched with hydroxylamine to a final concentration of 0.5% (v/v). Samples were combined, further desalted over StageTips, finally eluted into autosampler inserts (Thermo Scientific), dried in a speedvac and reconstituted with 5% Acetonitrile, 5% TFA for MS analysis. Mass spectrometric data were collected on an Orbitrap Eclipse mass spectrometer coupled to a Proxeon NanoLC-1000 UHPLC (Thermo Fisher Scientific). The 100 μm capillary column was packed in-house with 35 cm of Accucore 150 resin (2.6 μm, 150 Å; ThermoFisher Scientific). Data were acquired for 120 min per run. The scan sequence began with an MS1 spectrum: Orbitrap analysis, resolution 60,000, 400-1600 Th, automatic gain control (AGC) target set to Standard, automatic maximum injection time. MS2 analysis, which occurred in the Orbitrap, consisted of higher-energy collision dissociation (HCD), AGC 250%, NCE (normalized collision energy) 36, isolation window 0.5 Th, maximum injection time set to 86 ms and TopSpeed set at 1 sec. FAIMS compensation voltages (CVs) were set at −40V, −60V, and −80V. Database searching included all entries from the mouse UniProt Database (downloaded in May 2021). The database was concatenated with one composed of all protein sequences for that database in the reversed order [PMID: 17327847]. Raw files were converted to mzXML, and monoisotopic peaks were re-assigned using Monocle [PMID: 33190505]. Searches were performed with Comet [PMID: 23148064] using a 50-ppm precursor ion tolerance and fragment bin tolerance of 0.02. TMTpro labels on lysine residues and peptide N-termini +304.2071 Da, as well as carbamidomethylation of cysteine residues (+57.021 Da) were set as static modifications, while oxidation of methionine residues (+15.995 Da) was set as a variable modification. Peptide-spectrum matches (PSMs) were adjusted to a 1% false discovery rate (FDR) using a linear discriminant after which proteins were assembled further to a final protein-level FDR of 1% analysis [PMID: 21183079]. TMT reporter ion intensities were measured using a 0.003 Da window around the theoretical m/z for each reporter ion. Proteins were quantified by summing reporter ion counts across all matching PSMs. More specifically, reporter ion intensities were adjusted to correct for the isotopic impurities of the different TMTpro reagents according to manufacturer specifications. Peptides were filtered to exclude those with a summed signal-to-noise (SN)<160 across all TMT channels and <0.5 precursor isolation specificity. The signal-to-noise (S/N) measurements of peptides assigned to each protein were summed for a given protein.

Human ketone ester supplementation study. Following screening, eligible subjects participated in a randomized, counter-balanced, double-blind, placebo-controlled crossover trial comparing BHB supplementation versus placebo, separated by a minimum of 2 days (ClinicalTrials.gov ID NCT04194450). Pre-menopausal women were tested in the follicular phase (days 3-9). Participants were asked to refrain from structured exercise and alcohol, limit physical activity, and follow a standardized diet (~50% carbohydrate, ~30% fat, ~20% protein) on the day before each trial. After refraining from medication use for 24 hours, participants reported to the Exercise, Metabolism and Inflammation Laboratory at the University of British Columbia-Okanagan in an overnight fasted state. Anthropometric measurements were obtained prior to insertion of an indwelling intravenous catheter into the antecubital vein for repeated blood sampling. Blood was collected into EDTA tubes (BD, 367863) and immediately centrifuged at 2000 rpm for 15 min and the top layer of plasma was aliquoted and frozen at −80 °C. After baseline (0) blood sample collection, the oral BHB supplement or placebo was consumed in liquid form (~30-65 mL). The BHB supplement (H.V.M.N®, USA) in the form of the ketone monoester (R)-3-hydroxybutyl (R)-3-hydroxybutyrate (AG®, TDeltaS, Oxford, UK) contained 0.4 g mL-1 BHB, natural flavoring, and <2% stevia leaf extract. The taste-matched placebo drink contained the same natural flavoring as the ketone supplement, stevia leaf extract, 5 mL of Bittrex stock (0.005 g of denatonium benzoate powder in 40 ml of water) and 1.5 mL of arrowroot stock (2 g of arrowroot powder in 50 ml of water) to match the bitter flavor and viscosity of the ketone monoester. Supplements were dispensed into opaque 80 ml bottles with volume calculated for 0.3 g BHB/kg body weight and labelled either A or B by a third-party researcher. Blinding was maintained until completion of data collection and analyses.

Keto-Med study. Keto-Med was a single-site, randomized, crossover, interventional trial comparing 2 metabolically distinct diets (well-formulated ketogenic diet, WFKD; and Mediterranean-plus diet, Med-Plus) among individuals with prediabetes and T2DM. Forty participants aged ≥18 years with prediabetes or T2DM followed the WFKD and the Med-Plus for 12 weeks each, in random order. Participants were randomly assigned into 1 of 2 different diet sequences: WFKD for 12 weeks (phase 1) and then the Med-Plus for 12 weeks (phase 2), or the opposite order. Ad libitum intake was advised, and participants were guided to follow 2 sets of dietary guidelines that shared 3 important similarities (incorporating nonstarchy vegetables and avoiding added sugars and refined grains) and 3 important differences (incorporating compared with avoiding legumes, fruits, and whole, intact grains). During the WFKD phase, participants were counseled to sustain nutritional ketosis by limiting carbohydrates to 20-50 g/day and keeping proteins to ~1.5 g/kg ideal body weight/day, with the remaining kcals coming from fats. Participants were also instructed to consume >3 servings/day of nonstarchy vegetables and maintain adequate mineral and fluid intake for the ketogenic state (sodium, 3-5 g/day; potassium, 3-4 g/day). During the Med-Plus phase, participants were encouraged to sustain a Mediterranean diet based on recommendations from the Mediterranean Diet Pyramid, with the additional restriction of avoiding added sugars and refined grains. Instructions were to follow a mostly plant-based diet that included vegetables (including starchy vegetables); legumes; fruits; whole, intact grains; nuts; and seeds, with fish as the primary animal protein and olive oil as the primary fat. In both dietary phases, whole foods were promoted and all processed foods and added sugars were strongly discouraged. There was no prescribed washout period between intervention phases; therefore, participants immediately began either the WFKD (if on the Med-Plus first) or the Med-Plus (if on the WFKD first) for the second 12-week phase. During the first 4 weeks of each phase of the study, participants were provided, at no cost, with all meals and snacks. The 4 weeks of food delivery were followed by 8 weeks of the participants purchasing their own foods. To minimize the risk of hypoglycemia, individuals with T2DM were instructed to stop sulfonylurea medications before starting the WFKD and to reduce the dose by 50% before starting the Med-Plus. Sulfonylurea medications were restarted or increased for persistent hyperglycemia (>180 mg/dL).

Quantification and statistical analysis. Statistical analysis was performed in Prism 9.3.1. All data was expressed as mean±SEM unless otherwise specified. A student's two-sided t-test was used for pair-wise comparisons. Two-way ANOVA with repeated measures in one factor were used for time course data of repeated measurements. Unless otherwise specified, statistical significance was set as P<0.05. The specific test, P value symbol and error bar meaning, definition of center, and number of replicates are noted in figure legends.

Kaolin and water intake assays. DIO mice were individually housed and allowed ad libitum access to kaolin pellets for 5 days before the start of the experiment. Mice were also provided high-fat diet and had ad libitum access to water. On the day of the experiment, kaolin and high-fat diet pellets were replaced with fresh pellets. Mice were then injected with either vehicle or BHB-Phe (50 mg/kg, IP), and high-fat diet, kaolin and water intake were measured after 18 h.

Leptin, ghrelin and GDF-15 measurements. Plasma was collected from DIO mice 1 h after the administration of BHB-Phe (50 mg/kg, IP) or vehicle. ELISA kits for leptin (Crystal Chem 90030), ghrelin (Cayman Chemicals 10006307) and GDF-15 (R&D Systems DY6385-05) were used following manufacturer's instructions.

Tissue enzyme activity assays. Mouse tissues were mixed with cold PBS and homogenized using a Benchmark BeadBlaster Homogenizer at 4 °C. Protein concentrations were adjusted and the resulting crude was filtered through a 0.45-μm filter to remove insoluble materials. The in vitro reactions were conducted in Eppendorf tubes with 200 μg protein and incubation with the corresponding substrates at 37° C. for 1 hour. After that time, 10 μl of HCl and 400 μl of ethyl acetate were added in the BHB and lactate reactions and the samples were vortexed for 30 s. Reactions were centrifuged at 4° C. for 10 min at 15,000 r.p.m. and total of 300 μl from the top layer was transferred to a new Eppendorf tube and dried down under a stream of nitrogen. The residue was re-suspended in 100 μl of an 2:1:1 mixture of acetonitrile: methanol: water. The mixture was centrifuged at 4° C. for 10 min at 15,000 r.p.m. and the supernatant was transferred to a LC-MS vial. For carnosine reactions, the reactions were not acidified and were directly quenched with 300 μl of an 2:1:1 mixture of acetonitrile: methanol: water after the 1 h incubation and vortexed for 30 s. The mixture was centrifuged at 4° C. for 10 min at 15,000 r.p.m. and the supernatant was transferred to a LC-MS vial.

Recombinant CNDP2 purification. HEK293T cells were seeded in 15 cm plates at 11 million cells per plate and the next day transfected using polyfect with 27 μg of FLAG tagged mouse CNDP2. Cells were refreshed with new media the next day, and the day after collected for FLAG pulldown. On the day of collection, cells were washed 2 times with PBS and centrifuged to obtain the cell pellet. The pellet was re-suspended in PBS and sonicated. The whole cell lysate was then centrifuged for 30 minutes at 15,000 rpm at 4° C. to separate the membrane pellet and cytosolic supernatant. FLAG beads (Sigma, M8823) were washed 3 times with PBS then incubated with the cell supernatant overnight at 4° C. on rotation. After overnight incubation, the beads were washed 3 times with PBS before eluting with 0.1 mg/ml FLAG peptide in PBS (kept on rotation for 1 hour at room temperature). The concentration of recombinant CNDP2 was calculating using a FLAG blot with a protein standard curve.

Notwithstanding the appended claims, the disclosure is also defined by the following clauses:

1. A method of treating a metabolic disorder in a subject, the method comprising administering a therapeutically effective amount of an N-beta-hydroxybutyryl-amino acid to the subject to treat the subject for the metabolic disorder.

2. The method of clause 1, wherein the N-beta-hydroxybutyryl-amino acid comprises an amino acid with a hydrophobic side chain.

3. The method of clause 1 or 2, wherein the N-beta-hydroxybutyryl-amino acid is N-beta-hydroxybutyryl-phenylalanine (BHB-Phe), N-beta-hydroxybutyryl-leucine (BHB-Leu), N-beta-hydroxybutyryl-isoleucine (BHB-Ile), N-beta-hydroxybutyryl-valine (BHB-Val), or N-beta-hydroxybutyryl-methionine (BHB-Met).

4. The method of any one of clauses 1-3, wherein the metabolic disorder is obesity.

5. The method of any one of clauses 1-3, wherein the metabolic disorder is an obesity-related metabolic disorder.

6. The method of any one of clauses 1-3, wherein the metabolic disorder is diabetes.

7. The method of any one of clauses 1-6, wherein the method reduces food intake by the subject compared to food intake by the subject before treatment.

8. The method of clause 7, wherein cumulative food intake is reduced by 10% to 90%.

9. The method of clause 7 or 8, wherein average daily food intake is reduced by 10% to 90%.

10. The method of any one of clauses 7-9, wherein average daily food intake is reduced by at least 30%.

11. The method of any one of clauses 1-10, wherein the method reduces body weight of the subject compared to body weight of the subject before treatment.

12. The method of clause 11, wherein body weight is reduced by 1% to 50%.

13. The method of any one of clauses 1-12, wherein the method increases glucose clearance compared to glucose clearance of the subject before treatment.

14. The method of any one of clauses 1-13, wherein the method reduces adipose tissue mass compared to adipose tissue mass in the subject before treatment.

15. The method of clause 14, wherein the method reduces brown or inguinal fat by 30% to 50%.

16. The method of any one of clauses 1-15, wherein the method increases muscle mass compared to muscle mass in the subject before treatment.

17. The method of any one of clauses 1-16, wherein the method improves muscle function compared to muscle function in the subject before treatment.

18. The method of any one of clauses 1-17, wherein the method reduces inflammation compared to inflammation in the subject before treatment.

19. The method of any one of clauses 1-18, wherein health of the subject is rejuvenated compared to health of the subject before treatment.

20. The method of any one of clauses 1-19, wherein the method comprises administering the N-beta-hydroxybutyrl-amino acid for two or more consecutive days.

21. The method of any one of clauses 1-20, wherein the method comprises administering the N-beta-hydroxybutyryl-amino acid in combination with one or more therapies for treating the metabolic disorder.

22. The method of clause 21, wherein the one or more therapies comprise administering an active agent for treating the metabolic disorder.

23. The method of clause 21 or 22, wherein the one or more therapies comprise putting the subject on a low-calorie diet or increasing physical activity of the subject, or a combination thereof.

24. The method of any one of clauses 21-23, wherein the one or more therapies comprise surgical intervention.

25. The method of any one of clauses 21-24, wherein the one or more therapies comprise use of a weight loss device.

26. The method of any one of clauses 1-25, wherein the subject is an adult.

27. The method of any one of clauses 1-26, wherein the subject is a mammal.

28. The method of clause 27, wherein the subject is a human.

29. A composition comprising an N-beta-hydroxybutyryl-amino acid and an acceptable excipient.

30. The composition of clause 29, wherein the N-beta-hydroxybutyryl-amino acid comprises an amino acid with a hydrophobic side chain.

31. The composition of clause 29 or 30, wherein the N-beta-hydroxybutyryl-amino acid is N-beta-hydroxybutyryl-phenylalanine (BHB-Phe), N-beta-hydroxybutyryl-leucine (BHB-Leu), N-beta-hydroxybutyryl-isoleucine (BHB-Ile), N-beta-hydroxybutyryl-valine (BHB-Val), or N-beta-hydroxybutyryl-methionine (BHB-Met).

32. The composition of any one of clauses 29-31, wherein the N-beta-hydroxybutyryl-amino acid exhibits physiological activity.

33. The composition of clause 32, wherein the physiological activity is hypophagic activity.

34. A composition comprising an N-beta-hydroxybutyryl-amino acid for use in a method of treating a metabolic disorder.

35. The composition of clause 34, wherein the N-beta-hydroxybutyryl-amino acid comprises an amino acid with a hydrophobic side chain.

36. The composition of clause 34 or 35, wherein the N-beta-hydroxybutyryl-amino acid is N-beta-hydroxybutyryl-phenylalanine (BHB-Phe), N-beta-hydroxybutyryl-leucine (BHB-Leu), N-beta-hydroxybutyryl-isoleucine (BHB-Ile), N-beta-hydroxybutyryl-valine (BHB-Val), or N-beta-hydroxybutyryl-methionine (BHB-Met).

37. The composition of any one of clauses 34-36, wherein the metabolic disorder is obesity.

38. The composition of any one of clauses 34-36, wherein the metabolic disorder is an obesity-related metabolic disorder.

39. The composition of any one of clauses 34-36, wherein the metabolic disorder is diabetes.

40. A method of increasing muscle mass in a subject, the method comprising administering an effective amount of an N-beta-hydroxybutyryl-amino acid to the subject to increase muscle mass in the subject.

41. The method of clause 40, wherein the N-beta-hydroxybutyryl-amino acid comprises an amino acid with a hydrophobic side chain.

42. The method of clause 40 or 41, wherein the N-beta-hydroxybutyryl-amino acid is N-beta-hydroxybutyryl-phenylalanine (BHB-Phe), N-beta-hydroxybutyryl-leucine (BHB-Leu), N-beta-hydroxybutyryl-isoleucine (BHB-Ile), N-beta-hydroxybutyryl-valine (BHB-Val), or N-beta-hydroxybutyryl-methionine (BHB-Met).

43. The method of any one of clauses 40-42, wherein the method improves muscle function compared to muscle function in the subject before treatment.

44. A composition comprising an N-beta-hydroxybutyryl-amino acid for use in a method of increasing muscle mass and/or improving muscle function in a subject.

45. A method of treating an inflammatory disorder in a subject, the method comprising administering a therapeutically effective amount of an N-beta-hydroxybutyryl-amino acid to the subject to treat the inflammatory disorder in the subject.

46. The method of clause 45, wherein the N-beta-hydroxybutyryl-amino acid comprises an amino acid with a hydrophobic side chain.

47. The method of clause 45 or 46, wherein the N-beta-hydroxybutyryl-amino acid is N-beta-hydroxybutyryl-phenylalanine (BHB-Phe), N-beta-hydroxybutyryl-leucine (BHB-Leu), N-beta-hydroxybutyryl-isoleucine (BHB-Ile), N-beta-hydroxybutyryl-valine (BHB-Val), or N-beta-hydroxybutyryl-methionine (BHB-Met).

48. A composition comprising an N-beta-hydroxybutyryl-amino acid for use in a method of treating an inflammatory disorder in a subject.

49. A method of rejuvenating a subject, the method comprising administering an effective amount of an N-beta-hydroxybutyryl-amino acid to the subject to rejuvenate the subject.

50. The method of clause 49, wherein the N-beta-hydroxybutyryl-amino acid comprises an amino acid with a hydrophobic side chain.

51. The method of clause 49 or 50, wherein the N-beta-hydroxybutyryl-amino acid is N-beta-hydroxybutyryl-phenylalanine (BHB-Phe), N-beta-hydroxybutyryl-leucine (BHB-Leu), N-beta-hydroxybutyryl-isoleucine (BHB-Ile), N-beta-hydroxybutyryl-valine (BHB-Val), or N-beta-hydroxybutyryl-methionine (BHB-Met).

52. A composition comprising an N-beta-hydroxybutyryl-amino acid for use in a method of rejuvenating a subject.

53. A method of treating a muscle disorder in a subject, the method comprising administering a therapeutically effective amount of an N-beta-hydroxybutyryl-amino acid to the subject to treat the muscle disorder in the subject.

54. The method of clause 53, wherein the N-beta-hydroxybutyryl-amino acid comprises an amino acid with a hydrophobic side chain.

55. The method of clause 53 or 54, wherein the N-beta-hydroxybutyryl-amino acid is N-beta-hydroxybutyryl-phenylalanine (BHB-Phe), N-beta-hydroxybutyryl-leucine (BHB-Leu), N-beta-hydroxybutyryl-isoleucine (BHB-Ile), N-beta-hydroxybutyryl-valine (BHB-Val), or N-beta-hydroxybutyryl-methionine (BHB-Met).

56. A composition comprising an N-beta-hydroxybutyryl-amino acid for use in a method of treating a muscle disorder in a subject.

57. A method of decreasing body weight in a subject, the method comprising administering an effective amount of an N-beta-hydroxybutyryl-amino acid to the subject to decrease the body weight of the subject.

58. The method of clause 57, wherein the N-beta-hydroxybutyryl-amino acid comprises an amino acid with a hydrophobic side chain.

59. The method of clause 57 or 58, wherein the N-beta-hydroxybutyryl-amino acid is N-beta-hydroxybutyryl-phenylalanine (BHB-Phe), N-beta-hydroxybutyryl-leucine (BHB-Leu), N-beta-hydroxybutyryl-isoleucine (BHB-Ile), N-beta-hydroxybutyryl-valine (BHB-Val), or N-beta-hydroxybutyryl-methionine (BHB-Met).

60. The method of any one of clauses 57-59, wherein the method reduces food intake by the subject compared to food intake by the subject before administration of the N-beta-hydroxybutyryl-amino acid.

61. The method of clause 60, wherein cumulative food intake is reduced by 10% to 90%.

62. The method of clause 60 or 61, wherein average daily food intake is reduced by 10% to 90%.

63. The method of any one of clauses 60-62, wherein average daily food intake is reduced by at least 30%.

64. The method of any one of clauses 57-63, wherein the method comprises administering the N-beta-hydroxybutyryl-amino acid for two or more consecutive days.

65. A composition comprising an N-beta-hydroxybutyryl-amino acid for use in a method of decreasing body weight in a subject.

66. The composition of clause 65, wherein the N-beta-hydroxybutyryl-amino acid comprises an amino acid with a hydrophobic side chain.

67. The composition of clause 65 or 66, wherein the N-beta-hydroxybutyryl-amino acid is N-beta-hydroxybutyryl-phenylalanine (BHB-Phe), N-beta-hydroxybutyryl-leucine (BHB-Leu), N-beta-hydroxybutyryl-isoleucine (BHB-Ile), N-beta-hydroxybutyryl-valine (BHB-Val), or N-beta-hydroxybutyryl-methionine (BHB-Met).

68. A method of supplementing a subject's diet, the method comprising administering an effective amount of an N-beta-hydroxybutyryl-amino acid to the subject to supplement the subject's diet.

69. The method of clause 68, wherein the N-beta-hydroxybutyryl-amino acid comprises an amino acid with a hydrophobic side chain.

70. The method of clause 68 or 69, wherein the N-beta-hydroxybutyryl-amino acid is N-beta-hydroxybutyryl-phenylalanine (BHB-Phe), N-beta-hydroxybutyryl-leucine (BHB-Leu), N-beta-hydroxybutyryl-isoleucine (BHB-Ile), N-beta-hydroxybutyryl-valine (BHB-Val), or N-beta-hydroxybutyryl-methionine (BHB-Met).

71. The method of any one of clauses 68-70, wherein the method reduces food intake by the subject compared to food intake by the subject before treatment.

72. The method of clause 71, wherein cumulative food intake is reduced by 10% to 90%.

73. The method of clause 71 or 72, wherein average daily food intake is reduced by 10% to 90%.

74. The method of any one of clauses 71-73, wherein average daily food intake is reduced by at least 30%.

75. The method of any one of clauses 68-74, wherein the method reduces body weight of the subject compared to body weight of the subject before treatment.

76. The method of clause 75, wherein body weight is reduced by 1% to 50%.

77. The method of any one of clauses 68-76, wherein the method increases glucose clearance compared to glucose clearance of the subject before treatment.

78. The method of any one of clauses 68-77, wherein the method reduces adipose tissue mass compared to adipose tissue mass in the subject before treatment.

79. The method of clause 78, wherein the method reduces brown or inguinal fat by 30% to 50%.

80. The method of any one of clauses 68-79, wherein the method increases muscle mass compared to muscle mass in the subject before treatment.

81. The method of any one of clauses 68-80, wherein health of the subject is rejuvenated compared to health of the subject before treatment.

82. The method of any one of clauses 68-81, wherein the method comprises administering the N-beta-hydroxybutyrl-amino acid for two or more consecutive days.

83. A supplement composition comprising an N-beta-hydroxybutyryl-amino acid.

84. The supplement composition of clause 83, wherein the N-beta-hydroxybutyryl-amino acid comprises an amino acid with a hydrophobic side chain.

85. The supplement composition of clause 83 or 84, wherein the N-beta-hydroxybutyryl-amino acid is N-beta-hydroxybutyryl-phenylalanine (BHB-Phe), N-beta-hydroxybutyryl-leucine (BHB-Leu), N-beta-hydroxybutyryl-isoleucine (BHB-Ile), N-beta-hydroxybutyryl-valine (BHB-Val), or N-beta-hydroxybutyryl-methionine (BHB-Met).

86. The supplement composition of any one of clauses 83-85, wherein the N-beta-hydroxybutyryl-amino acid exhibits physiological activity.

87. The supplement composition of clause 86, wherein the physiological activity is hypophagic activity.

In at least some of the previously described embodiments, one or more elements usedin an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

What is claimed is:

1. A method of treating a metabolic disorder in a subject, the method comprising administering a therapeutically effective amount of an N-beta-hydroxybutyryl-amino acid to the subject to treat the subject for the metabolic disorder.

2. The method of claim 1, wherein the N-beta-hydroxybutyryl-amino acid comprises an amino acid with a hydrophobic side chain.

3. The method of claim 1 or 2, wherein the N-beta-hydroxybutyryl-amino acid is N-beta-hydroxybutyryl-phenylalanine (BHB-Phe), N-beta-hydroxybutyryl-leucine (BHB-Leu), N-beta-hydroxybutyryl-isoleucine (BHB-Ile), N-beta-hydroxybutyryl-valine (BHB-Val), or N-beta-hydroxybutyryl-methionine (BHB-Met).

4. The method of any one of claims 1-3, wherein the metabolic disorder is obesity.

5. The method of any one of claims 1-3, wherein the metabolic disorder is an obesity-related metabolic disorder.

6. The method of any one of claims 1-3, wherein the metabolic disorder is diabetes.

7. The method of any one of claims 1-6, wherein the method reduces food intake by the subject compared to food intake by the subject before treatment.

8. The method of any one of claims 1-7, wherein the method reduces body weight of the subject compared to body weight of the subject before treatment.

9. The method of any one of claims 1-8, wherein the method increases glucose clearance compared to glucose clearance of the subject before treatment.

10. The method of any one of claims 1-9, wherein the method reduces adipose tissue mass compared to adipose tissue mass in the subject before treatment.

11. The method of any one of claims 1-10, wherein the method increases muscle mass compared to muscle mass in the subject before treatment.

12. A method of decreasing body weight in a subject, the method comprising administering an effective amount of an N-beta-hydroxybutyryl-amino acid to the subject to decrease the body weight of the subject.

13. A composition comprising an N-beta-hydroxybutyryl-amino acid for use in a method of decreasing body weight in a subject.

14. A method of supplementing a subject's diet, the method comprising administering an effective amount of an N-beta-hydroxybutyryl-amino acid to the subject to supplement the subject's diet.

15. A supplement composition comprising an N-beta-hydroxybutyryl-amino acid.
